SEQUENCE LISTING (5 sequences)

SEQ ID NO: 1 - DNA, 21 bp, Artificial Sequence
  Synthetic Polynucleotide
cggccugagcguccagaaatt

SEQ ID NO: 2 - DNA, 19 bp, Artificial Sequence
  Synthetic Polynucleotide
tgtgcaagctcagcttgga

SEQ ID NO: 3 - DNA, 21 bp, Artificial Sequence
  Synthetic Polynucleotide
aggtgtcttcaataggcccaa

SEQ ID NO: 4 - DNA, 19 bp, Artificial Sequence
  Synthetic Polynucleotide
gaaggtgaaggtcggagtc

SEQ ID NO: 5 - DNA, 20 bp, Artificial Sequence
  Synthetic Polynucleotide
gaagatggtgatgggatttc

(19) United States
(12) Patent Application Publication
(10) Pub. No.: US 2017/0097358 A1
(43) Pub. Date: Apr. 6, 2017

(54) EARLY DETECTION OF PREECLAMPSIA

(71) Applicant: Igenomix S.L., Valencia (ES)

(72) Inventors: Carlos Simón, Valencia (ES); Tamara Garrido, Valencia (ES); Antonio Pellicer, Paterna (ES)

(73) Assignee: Igenomix S.L., Valencia (ES)

(21) Appl. No.: 15/127,901

(22) PCT Filed: Mar. 19, 2015
(86) PCT No.: PCT/IB2015/001404

§ 371 Date: Sep. 21, 2016

Related U.S. Application Data
(60) Provisional application No. 61/969,520, filed on Mar. 24, 2014.
(60) Provisional application No. 61/968,728, filed on Mar. 21, 2014.

(51) Int. Cl. G01N 33/68; A61K 31/727

(52) U.S. Cl. CPC G01N 33/689; A61K 31/727; G01N 2800/368; G01N 2333/47

(57) ABSTRACT

The invention provides non-invasive assays to reliably identify women who have or are predisposed to developing preeclampsia (PE). The method comprises measuring a level of annexin A2 (ANXA2) in a test sample obtained of a subject; and identifying the subject as having preeclampsia or at an increased risk of developing preeclampsia when the level of ANXA2 in the test sample is decreased in relation to a control sample. Methods to treat subject identified as having PE or at an increased risk of developing preeclampsia are also provided.

### RELATED APPLICATIONS

This application claims the benefit under 35 U.S.C. §119(e) of U.S. provisional application Ser. No. 61/968,728, filed Mar. 21, 2014, and U.S. provisional application Ser. No. 61/969,520, filed Mar. 24, 2014, the contents of which are incorporated by reference herein in its entirety.

### FIELD OF THE INVENTION

The present invention generally relates to biomarkers for preeclampsia as well as methods for treating this disease.

### BACKGROUND OF THE INVENTION

Preeclampsia (PE) is a leading cause of maternal and fetal morbidity and mortality affecting 4%-8% of pregnancies, leading to over 8 million cases worldwide per year. Clinically preeclampsia is defined by the existence of high blood pressure, proteinuria, edema, and, in some patients, HELLP syndrome and eclampsia. Extensive efforts have been made to develop markers that can accurately predict preeclampsia. Biochemical markers and Doppler ultrasound measurements of blood flow in the maternal uterine arteries have been tested extensively but none of these has thus far achieved widespread clinical use (Conde-Agudelo et al., Obstet General 2004; 104: 1367-91). There remains a need to develop reliable and clinically useful markers for predicting preeclampsia. Being able to identify pregnant women at risk for developing preeclampsia could permit the use of prophylactic agents which are known to be effective in preventing preeclampsia development.

### SUMMARY OF THE INVENTION

The present invention provides non-invasive assays to reliably identify women who are predisposed to developing PE. This allows for early intervention with appropriate therapy to prevent or attenuate PE. The present invention is based, at least in part, on the discovery that endometrial annexin A2 (ANXA2) levels are decreased in women that had preeclampsia (PE) in their previous pregnancies as compared to the levels in women who had normal (healthy) pregnancies.

According to some aspect of the invention, a method for treating preeclampsia is provided. The method comprises determining whether a subject is at an increased risk of developing preeclampsia by measuring a level of annexin A2 (ANXA2) in a test sample obtained of the subject, comparing the level of ANXA2 in the test sample to a control level of ANXA2 to determine if the subject is at an increased risk of developing preeclampsia; and administering to the subject determined to be at an increased risk of developing preeclampsia an effective amount of a glycosaminoglycan.

In some embodiments, the subject has no history of preeclampsia. In some embodiments, the sample is selected from the group consisting of a sample of endometrium tissue, endometrial stromal cells and endometrial fluid. In some embodiments, the control level of ANXA2 is derived from subjects who have had a successful pregnancy and no history of preeclampsia. In some embodiments, the glycosaminoglycan is selected from the group consisting of low molecular weight heparin, heparan sulfate, chemically modified heparin or heparan sulfate, low molecular weight dermatan sulfates and mixtures thereof. In some embodiments, the level of ANXA2 is determined using an immune assay selected from the group consisting of ELISA, Western Blot, and immunohistochemical staining. In some embodiments, the subject is known to be pregnant. In some embodiments, the subject is trying to get pregnant.

Some aspects of the invention provide a method for diagnosing preeclampsia or aiding in the diagnosis of preeclampsia. The method comprises measuring a level of annexin A2 (ANXA2) in a test sample obtained of a subject; and comparing the level of ANXA2 in the test sample to a control level of ANXA2 to determine if the subject is at an increased risk of developing preeclampsia.

In some embodiments, the subject has no history of preeclampsia. In some embodiments, the sample is selected from the group consisting of a sample of endometrium tissue, endometrial stromal cells and endometrial fluid. In some embodiments, the control sample is obtained from subjects who have had a successful pregnancy and no history of preeclampsia. In some embodiments, the level of ANXA2 is determined using an immune assay selected from the group consisting of ELISA, Western Blot, and immunohistochemical staining. In some embodiments, the subject is known to be pregnant. In some embodiments, the subject is trying to get pregnant.

Some aspects of the invention provide a method for treating preeclampsia. The method comprises obtaining an endometrial fluid sample of a subject who does not presently have preeclampsia, wherein the subject is pregnant or wherein the subject has plans to become pregnant; performing an assay to determine level of ANXA2 in the endometrial fluid sample; comparing the level of ANXA2 in the endometrial fluid sample to a control level of ANXA2 to determine if the subject is at an increased risk of developing preeclampsia; and administering to the subject an effective amount of a glycosaminoglycan if the subject is determined to be at an increased risk of developing preeclampsia.

In some embodiments, the subject has no history of preeclampsia. In some embodiments, the control level of ANXA2 is derived from subjects who have had a successful pregnancy and no history of preeclampsia. In some embodiments, the glycosaminoglycan is selected from the group consisting of low molecular weight heparin, heparan sulfate, chemically modified heparin or heparan sulfate, low molecular weight dermatan sulfates and mixtures thereof. In some embodiments, the level of ANXA2 is determined using an immune assay selected from the group consisting of ELISA, Western Blot, and immunohistochemical staining.

Some aspects of the invention provide a method for treating preeclampsia. The method comprises identifying a subject that has low levels of ANXA2 as compared to a control level of ANXA2, has plans to get pregnant and has no history of preeclampsia; and administering to the subject a glycosaminoglycan in amount sufficient to raise the level of ANXA2 in the subject.

In some embodiments, the control level of ANXA2 is derived from subjects who have had a successful pregnancy and no history of preeclampsia. In some embodiments, the glycosaminoglycan is selected from the group consisting of low molecular weight heparin, heparan sulfate, chemically modified heparin or heparan sulfate, low molecular weight dermatan sulfates and mixtures thereof. In some embodiments, the level of ANXA2 is determined using an immune assay selected from the group consisting of ELISA, Western Blot, and immunohistochemical staining.

Some aspects of the invention provide a method for assessing efficacy of glycosaminoglycan therapy for preeclampsia. The method comprises treating a subject who has or is at an increased risk of developing preeclampsia an effective amount of a glycosaminoglycan; measuring levels of annexin A2 (ANXA2) in test samples obtained of the subject before and after the treatment with glycosaminoglycan, wherein an increase in the level of ANXA2 after treatment in relation to the level before treatment indicates that the glycosaminoglycan therapy is effective.

In some embodiments, the glycosaminoglycan is selected from the group consisting of low molecular weight heparin, heparan sulfate, chemically modified heparin or heparan sulfate, low molecular weight dermatan sulfates and mixtures thereof. In some embodiments, the level of ANXA2 is determined using an immune assay selected from the group consisting of ELISA, Western Blot, and immunohistochemical staining.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

### BRIEF DESCRIPTION OF THE DRAWINGS

FIG. 1 shows in vitro hESC decidualization in patients that have suffered sPE in previous pregnancies. FIGS. 1A and 1B show Prolactin and IGFBP-1 secretions measured by ELISA on decidual vs. non-decidual hESC from women that have suffered severe preeclampsia (sPE) in their previous pregnancies (n=13) and control patients (non-PE) (n=13). Prolactin and IGFBP-1 secretions were presented as ng/ml (mean±sd) in non-decidual (black bar) and decidualized (grey bar) and media values were schematized on graph. FIG. 1B shows IGFBP-1 secretions measured by ELISA. FIG. 1C shows F-actin remodeling when in vitro decidualization was induced on hESC from sPE and non-PE compared with non-decidual hESCs. *, P<0.05; **, P<0.005

FIG. 2 shows immunohistochemistry and western blot analysis of ANXA2 in sPE. FIG. 2A shows total cellular proteins extracted from biopsies of severe preeclampsia (sPE) endometria which were subjected to SDS-PAGE, and immunoblotted with ANXA2 antibody and housekeeping protein, β-actin. Densitometric analyses of ANXA2 was performed from 3 different experiments and normalized with GAPDH. FIG. 2B shows the staining profile of ANXA2 content observed in non-PE and sPE endometrial tissue. FIG. 2C shows ANXA2 western blot and densitometric analysis of total cellular protein extract obtained from hESC decidualized and non-decidualized endometria of sPE and non-PE patients. FIG. 2D shows intracellular and extracellular ANXA2 analysis of protein extract and conditioned media hESCs, respectively. ANXA2 protein was measured by ELISA and expressed as ng/mL (mean±sd) from three different experiments. *, P<0.05; **, P<0.005

FIG. 3 shows the effect of ANXA2 inhibition on in vitro decidualization. FIG. 3A shows ANXA2 western blot and densitometry analysis of decidual hESC by two systems: P4+E2 and cAMP+MPA compared with non-treated hESC. FIG. 3B shows the extracellular ANXA2 level on conditioned media of decidualized and non-treated hESC measured by ELISA in three different experiments. mRNA (FIG. 3C) and protein ANXA2 levels (FIG. 3D) of control cells (nontransfected), cells transfected with a scramble sequence (control siRNA), or cells transfected with an ANXA2-specific siRNA (ANXA2 siRNA) were evaluated by RT-PCR and western blot analysis. FIGS. 3E and 3F show PRL and IGFBP-1 levels measured by ELISA on conditioned media of controls and ANXA2 siRNA inhibited hESC. FIG. 3G shows F-actin architecture in the control, control siRNA and ANXA2-inhibited hESCs, visualized by a rhodamine phalloidin stain. FIG. 3H shows G-actin (soluble), F-actin (filamentous), and total actin fractions analyzed by an in vivo assay, and the results were observed by western blot analysis in the ANXA2-inhibited and control hESCs. Densitometric analysis was performed from 3 different experiments, expressed as the G/F actin ratio, and normalized with total actin.

FIG. 4 shows motility, trophoblast spreading and invasion analysis of ANXA2 inhibited hESCs. FIG. 4A shows a wound-healing assay on controls and ANXA2-inhibited hESCs. Wound width was measured at 0 and 24 h after wounding. Percentage of wound closure was determined by an image analysis. Values are means of 10 measurements from 3 different experiments. FIG. 4B shows hESCs transfected with ANXA2 siRNA and then cocultured with mouse blastocyst until embryo attachment occurred. After 48 h, hESCs were immunostained with vimentin and mouse trophoblast cells with E-cadherin. Mouse blastocyst spreading on hESCs was encircled with white line and area was measured in pixels. FIG. 4C shows a schematic representation of the collagen transwell invasion assay used to measure the effect of human trophoblast JEG-3 cell invasion on ANXA2 inhibited hESCs. Histograms show the percentage of the JEG-3 invading cells, with invasion of the control cells designated as 100%. Data represent the mean of three independent experiments. *, P<0.05; **, P<0.005

FIG. 5 shows fibrinolytic activity on ANXA2 inhibited hESCs and sPE hESCs. FIG. 5A shows plasminogen level on conditioned media of ANXA2 inhibited hESC and hESC from sPE patients evaluated by ELISA in three different experiments and expressed as media values pg/mL. FIG. 5B shows plasmin activity present with hESC conditioned media evaluated by fluorometric functional assay and expressed as mM concentration of active plasmin. MMP2 (FIG. 5C) and MMP9 (FIG. 5D) protein levels were evaluated on conditioned media of ANXA2 inhibited hESCs and sPE hESCs by ELISA in three different experiments. FIG. 5E shows control, control siRNA, ANXA2 siRNA and sPE hESCs treated with 50 or 100 μg/mL of heparin and analyzed for plasminogen level during 0, 15, 30 and 60 minute intervals and also without treatment. FIG. 5E shows plasmin activity measured on conditioned media of ANXA2 inhibited hESCs and sPE hESCs treated with or without 100 μg/mL of heparin. FIG. 5F shows ANXA2 protein secreted on conditioned media of hESC treated with heparin dose. MMP2 (FIG. 5H) and MMP9 (FIG. 5G) levels were evaluated by ELISA on conditioned media of heparin treated hESCs.

FIG. 6 shows a model that integrates hESC decidualization resistance present in sPE, mediated at least in part by ANXA2 deficiency, with shallow trophoblast invasion and fibrinolytic alterations as a maternal cause of PE.

FIG. 7 shows a study of ANXA2 levels in endometrial fluid.

### DETAILED DESCRIPTION OF THE INVENTION

The present invention is based, at least in part, on the discovery that endometrial annexin A2 (ANXA2) levels are decreased in women that had preeclampsia (PE) in their previous pregnancies as compared to the levels in women who had normal pregnancies. The methods of the invention provide non-invasive assays to reliably identify women who are predisposed to developing PE. Thus, the instant invention enables the early detection of a predisposition to developing PE before symptoms develop, thereby permitting appropriate therapy to be initiated in a timely fashion. Another advantage of the present invention is that women who have been determined to be at an increased risk for preeclampsia can be treated with agents that increase ANXA2 levels so as to prevent or attenuate preeclampsia.

Preeclampsia (PE) is a condition characterized by high blood pressure (systolic blood pressure≧140 mmHg and/or diastolic blood pressure≧90 mmHg) occurring after 20 weeks of pregnancy in women with previously normal blood pressure. In addition there is an increased level of proteins in the urine compared to normal. Increased proteinuria is defined as ≧300 mg in a 24 hour collection of urine (The National High Blood Pressure Education Program Working Group Report on High Blood Pressure in Pregnancy. Am J Obstet General 2000; 183: S1-S22). Along with elevated blood pressure, there may be associated signs and symptoms such as headache, abdominal pain, bleeding problems, seizure and complications, such as poor fetal growth, preterm birth and even death of the fetus or mother. The frequency is 5-8% of all pregnancies but can be much greater in certain groups, e.g. women carrying twins.

According to one aspect of the invention, a method for treating preeclampsia is provided. The method comprises determining whether a subject has or is at an increased risk of developing preeclampsia by measuring a level of annexin A2 (ANXA2) in a test sample obtained of the subject, comparing the level of ANXA2 in the test sample to a control level of ANXA2 to determine if the subject has or is at an increased risk of developing preeclampsia; and administering to the subject determined to be at an increased risk of developing preeclampsia an effective amount of an agent known to raise the levels of ANXA2.

In some embodiments, the method comprises determining whether a subject is at an increased risk of developing preeclampsia by measuring a level of annexin A2 (ANXA2) in a test sample obtained of the subject, comparing the level of ANXA2 in the test sample to a control level of ANXA2 to determine if the subject is at an increased risk of developing preeclampsia; and administering to the subject determined to be at an increased risk of developing preeclampsia an effective amount of a glycosaminoglycan.

According to one aspect of the invention, a method for diagnosing preeclampsia or aiding in the diagnosis of preeclampsia is provided. The method comprises measuring a level of annexin A2 (ANXA2) in a test sample obtained of a subject; and comparing the level of ANXA2 in the test sample to a control level of ANXA2 to determine if the subject is at an increased risk of developing preeclampsia.

According to one aspect of the invention, a method for treating preeclampsia. The method comprises obtaining an endometrial fluid sample of a subject who does not presently have preeclampsia, wherein the subject is pregnant or wherein the subject has plans to become pregnant; performing an assay to determine level of ANXA2 in the endometrial fluid sample; comparing the level of ANXA2 in the endometrial fluid sample to a control level of ANXA2 to determine if the subject is at an increased risk of developing preeclampsia; and administering to the subject an effective amount of a glycosaminoglycan if the subject is determined to be at an increased risk of developing preeclampsia.

According to one aspect of the invention, a method for treating preeclampsia is provided. The method comprises identifying a subject that has low levels of ANXA2 as compared to a control level of ANXA2, plans to get pregnant and has no history of preeclampsia; and administering to the subject a glycosaminoglycan in amount sufficient to raise the level of ANXA2 in the subject.

According to one aspect of the invention, a method for assessing efficacy of glycosaminoglycan therapy for preeclampsia is provided. The method comprises treating a subject who has or is at an increased risk of developing preeclampsia an effective amount of a glycosaminoglycan; measuring levels of annexin A2 (ANXA2) in test samples obtained of the subject before and after the treatment with glycosaminoglycan, wherein an increase in the level of ANXA2 after treatment in relation to the level before treatment indicates that the glycosaminoglycan therapy is effective.

As used herein, “a subject” includes all mammals, including, but not limited to, dogs, cats, horses, sheep, goats, cows, pigs, humans, and non-human primates. In some embodiments, the subject is a woman. As used herein, a subject “at increased risk for developing preeclampsia” includes a subject who has a higher probability of developing preeclampsia when compared to an average representative of the population. In some embodiments, the subject is known to be pregnant. In some embodiments, the subject is trying to get pregnant. The subject may have had no previous pregnancies, one or more normal previous pregnancies or has suffered PE in a previous pregnancy. In some embodiments, the subject has one or more risk factors for preeclampsia. For example the subject may have one or any combination of the following: the subject is pregnant with more than one baby, has a history of chronic high blood pressure, diabetes, kidney disease or organ transplant, is pregnant for the first time, is obese, particularly with Body Mass Index (BMI) of 30 or greater, is over the age of 40 or under the age of 18 years, has a family history of preeclampsia (i.e., a mother, sister, grandmother or aunt had the disorder), has polycystic ovarian syndrome, has Lupus or other autoimmune disorders, including rheumatoid arthritis, sarcoidosis and multiple sclerosis, has had in-vitro fertilization or has sickle cell disease.

In some embodiments, the methods described herein, comprise identifying a subject that has low levels of ANXA2 as compared to a control level of ANXA2, plans to get pregnant and has no history of preeclampsia. As used herein, “identifying a subject that has low levels of ANXA2 as compared to a control level of ANXA2, plans to get pregnant and has no history of preeclampsia” means selecting a subject that has low levels of ANXA2 as compared to a control level of ANXA2, has plans to get pregnant and has no history of preeclampsia. The subject so identified or selected is treated for PE by administering to the subject a glycosaminoglycan in amount sufficient to raise the level of ANXA2 in the subject.

The term “test sample” refers to a sample derived from a subject being evaluated using a method of the invention, e.g., a subject who is pregnant or trying to get pregnant. Non-limiting examples of the sample include endometrium tissue, endometrial stromal cells and endometrial fluid. Obtaining a sample of a subject means taking possession of a sample of the subject. Obtaining a sample from a subject means removing a sample from the subject. Therefore, the person obtaining a sample of a subject and measuring a level of ANXA2 in the sample does not necessarily obtain the sample from the subject. In some embodiments, the sample may be removed from the subject by a medical practitioner (e.g., a doctor, nurse, or a clinical laboratory practitioner), and then provided to the person measuring a level of ANXA2. The sample may be provided to the person measuring a level of ANXA2 by the subject or by a medical practitioner (e.g., a doctor, nurse, or a clinical laboratory practitioner). In some embodiments, the person measuring a level of ANXA2 obtains a sample from the subject by removing the sample from the subject.

Annexin A2 (ANXA2) is a calcium-regulated phospholipid binding protein that is significantly up-regulated during the mid- and late-secretory phases of the human endometrium. This protein is key to the acquisition of the receptive phenotype by the endometrial epithelium by the modulation the F-actin network. ANXA2 is a pro-fibrinolytic receptor, present and functional on human endometrial stromal cells (hESC). It acts as a cell surface co-receptor for plasminogen and its activator tPA, enhancing significantly cell surface plasmin generation. As used herein the term “Annexin A2” refers to any known isoform of Annexin A2. Without being so limited, it includes nucleic acid sequences NM_001002858.2, NM_001136015.2, NM_004039.2, and NM_001002857.1 and protein sequences NP_001002858.1, NP_001129487.1, NP_004030.1 and NP_001002857.1. Other known Annexin A2 nucleic acid and encoded polypeptides are described in WO 2009/143633 (incorporated by reference herein).

The methods disclosed herein typically comprise measuring a level of ANXA2 in a sample or performing an assay to determine the level of ANXA2. Levels of ANXA2 may in general be detected by either detecting mRNA from the cells and/or detecting expression products, such as polypeptides and proteins. Expression of the transcripts and/or proteins encoded by the nucleic acids may be measured by any of a variety of known methods in the art. For example, methods to measure the level of ANXA2 proteins include, but are not limited to, enzyme-linked immunosorbant assay (ELISA), Western blot, immunohistochemical analysis, radioimmunoassay (RIA), mass spectrometry, microarray, and microscopy. Methods to detect ANXA2 nucleic acid sequences include, but are not limited to, polymerase chain reaction (PCR), reverse transcriptase-PCR (RT-PCR), in situ PCR, quantitative PCR (q-PCR), in situ hybridization, Southern blot, Northern blot, sequence analysis, microarray analysis, detection of a reporter gene, or other DNA/RNA hybridization platforms.

The methods disclosed herein typically comprise comparing the level of ANXA2 in the test sample to a control level of ANXA2 to determine if the subject is at an increased risk of developing preeclampsia. In some embodiments, the “control level of ANXA2” is derived from subjects who have had a successful pregnancy and no history of preeclampsia. In such instances, when the control level of ANXA2 is derived from subjects who have had a successful pregnancy and no history of preeclampsia, a level of ANXA2 in the test sample lower than the control level is indicative that the subject has or is at an increased risk of developing preeclampsia. In some embodiments, the control level is known to be predictive of developing PE, and in such instances, a level of ANXA2 in the test sample that corresponds to the control level indicates that the subject has or is at an increased risk of developing preeclampsia. Thus, rather than determining whether a test level is statistically lower than a control level, one could determine whether the test level is within a range known to be predictive of developing PE. The control level may be a fixed number, for example, in ANXA2 units per ml of endometrial fluid. The control level may be a range. The control level may be a comparative level measured in a control sample, the level being measured simultaneously with the assay of the test level. The control level may be expressed as an average with a standard deviation. The invention is not intended to be limited by the particular methodology by which a test sample is determined to be statistically lower than or corresponds to a control.

In some embodiments, the ANXA2 levels are measured in any phase throughout the menstrual cycle. In some embodiments, the levels of ANXA2 are measured during the luteal phase of the menstrual cycle. In some embodiments, the levels of ANXA2 are measured during the mid luteal phase (days 18-24) of the menstrual cycle. In some embodiments, the average control endometrial fluid level of ANXA2 measured in normal subjects (i.e., subjects who have had a successful pregnancy and no history of preeclampsia) in the mid luteal phase (days 18-24) of the menstrual cycle is 32 μg/ml (mean±4), while the level of ANXA2 in the mid luteal phase in subjects predisposed to PE is significantly reduced as compared to this control level. In some embodiments, the level of ANXA2 in subjects predisposed to PE is 2, 3, 4, or 5 standard deviations lower than this average control ANXA2 level. In some embodiments, the level of ANXA2 in the mid luteal phase in subjects predisposed to PE is less than 5, less than 10, less than 15, less than 20, or less than 25 μg/ml.

In some embodiments, a decrease in the level of ANXA2 in the test sample in relation to a control sample is indicative that the subject has or is at an increased risk of developing preeclampsia. By “decreased expression” it means that the expression of ANXA2 in the test sample has a statistically significant decrease from that in the control sample. For example, a significant decrease may be detected when the expression level of ANXA2 in the test sample is at least 1%, at least 5%, at least 10%, at least 25%, at least 50%, at least 100%, at least 250%, at least 500%, or at least 1000% lower, than that in the control sample. Similarly, a significant decrease may be detected when the expression level of ANXA2 in the test sample is at least 2-fold, at least 3-fold, at least 4-fold, at least 5-fold, at least 6-fold, at least 7-fold, at least 8-fold, at least 9-fold, at least 10-fold, at least 20-fold, at least 30-fold, at least 40-fold, at least 50-fold, at least 100-fold, or more lower, than that of a control sample. Significant differences may be identified by using an appropriate statistical test. Tests for statistical significance are well known in the art and are exemplified in Applied Statistics for Engineers and Scientists by Petruccelli, Chen and Nandram 1999 Reprint Ed.

In some embodiments, a report summarizing the results of the analysis, i.e. whether the subject has or is predisposed to have PE and any other information pertaining to the analysis could optionally be generated as part of the analysis (which may be interchangeably referred to herein as “providing” a report, “producing” a report, or “generating” a report). For example, measurements of blood pressure, and/or protein content in urine may be determined, and these may be included in the report. Examples of reports may include, but are not limited to, reports in paper (such as computer-generated printouts of test results) or equivalent formats and reports stored on computer readable medium (such as a CD, computer hard drive, or computer network server, etc.). Reports, particularly those stored on computer readable medium, can be part of a database (such as a database of patient records, which may be a “secure database” that has security features that limit access to the report, such as to allow only the patient and the patient's medical practitioners to view the report, for example). In addition to, or as an alternative to, generating a tangible report, reports can also be displayed on a computer screen (or the display of another electronic device or instrument).

A report can further be transmitted, communicated or reported (these terms may be used herein interchangeably), such as to the individual who was tested, a medical practitioner (e.g., a doctor, nurse, clinical laboratory practitioner, genetic counselor, etc.), a healthcare organization, a clinical laboratory, and/or any other party intended to view or possess the report. The act of ‘transmitting’ or ‘communicating’ a report can be by any means known in the art, based on the form of the report, and includes both oral and non-oral transmission. Furthermore, “transmitting” or “communicating” a report can include delivering a report (“pushing”) and/or retrieving (“pulling”) a report. For example, non-oral reports can be transmitted/communicated by such means as being physically transferred between parties (such as for reports in paper format), such as by being physically delivered from one party to another, or by being transmitted electronically or in signal form (e.g., via e-mail or over the internet, by facsimile, and/or by any wired or wireless communication methods known in the art), such as by being retrieved from a database stored on a computer network server, etc.

In some embodiments, the methods described herein, comprise treating subjects who are identified as having or being predisposed to developing preeclampsia with an effective amount of an agent that is known to increase ANXA2 levels so as to prevent or attenuate preeclampsia. Agents known to increase ANXA2 levels include, but are not limited to, a glycosaminoglycan. Examples of a glycosaminoglycan include, but are not limited to, low molecular weight heparin, heparan sulfate, chemically modified heparin or heparan sulfate, low molecular weight dermatan sulfates and mixtures thereof. Additional examples of glycosaminoglycans for the treatment for preeclampsia are described in EP 1016410, incorporated herein by reference.

As used herein, the term “treat” means to reduce or ameliorate the risk of a subject developing PE. A reduction in the risk of a subject developing PE may be manifest as an increase in the levels of ANXA2 as compared to ANXA2 levels obtained before treatment or as compared to control normal ANXA2 levels (i.e., ANXA2 levels of subject who have has normal previous pregnancies and no history of PE). In some embodiments, the term “treat” means to reduce or ameliorate PE by a detectable amount or degree. The term “treat” as used herein refers to both complete and partial treatment. For example, treating PE may be manifest as a reduction in protein levels in urine and/or a decrease in blood pressure levels as compared to blood pressure levels obtained before treatment or as compared to control normal blood pressure levels.

An “effective amount” of a glycosaminoglycan refers to an amount sufficient to elicit the desired biological response, i.e., treating the preeclampsia. As will be appreciated by those of ordinary skill in this art, the effective amount of glycosaminoglycan may vary depending on such factors as the desired biological endpoint, the pharmacokinetics of the compound, the condition being treated, the mode of administration, and the age and health of the subject. An effective amount includes, but is not limited to, that amount necessary to slow, reduce, inhibit, ameliorate or reverse one or more symptoms associated with PE. In the treatment of PE, such amount may refer to an amount sufficient to decrease blood pressure levels as compared to blood pressure levels obtained before treatment or as compared to control normal blood pressure levels. In some embodiments, an effective amount may refer to an amount sufficient to cause a reduction in protein levels in urine. In some embodiments, an effective amount may refer to an amount sufficient to reduce the risk of a subject developing PE. Such amount may refer to an amount sufficient to increase/raise the levels of ANXA2 as compared to ANXA2 levels obtained before treatment or as compared to control normal ANXA2 levels (i.e., ANXA2 levels of subjects who have had a successful pregnancy and no history of preeclampsia). In some embodiments, the amount is sufficient to re-establish control normal levels of ANXA2 in the treated subject.

An effective amount of a compound may vary from about 0.001 mg/kg to about 1000 mg/kg in one or more dose administrations, for one or several days (depending on the mode of administration). In certain embodiments, the effective amount varies from about 0.001 mg/kg to about 1000 mg/kg, from about 0.01 mg/kg to about 750 mg/kg, from about 0.1 mg/kg to about 500 mg/kg, from about 1.0 mg/kg to about 250 mg/kg, and from about 10.0 mg/kg to about 150 mg/kg. In some embodiments, the effective amount is 1000, 2000, 3000, 40000, 5000, 6000, or 7000 IU of glycosaminoglycan. In some embodiments, the effective amount is 5000 IU of glycosaminoglycan, (e.g., low molecular weight heparin). The glycosaminoglycan can be administered via any suitable route of administration. For example, the glycosaminoglycan can be administered via subcutaneous, intravenous, intraperitoneal, or intramuscular routes.

In some embodiments, the methods described herein comprise measuring levels of annexin A2 (ANXA2) in test samples obtained of the subject before and after the treatment with glycosaminoglycan. An effective therapy is expected to increase the level of ANXA2 after treatment in relation to the level before treatment. Thus, an effective therapy is indicated by an increase in the level of ANXA2 after treatment.

The present invention is further illustrated by the following Examples, which in no way should be construed as further limiting. The entire contents of all of the references (including literature references, issued patents, published patent applications, and co pending patent applications) cited throughout this application are hereby expressly incorporated by reference.

### EXAMPLES

### Example 1

### Endometrial Decidualization Resistance Mediated Through ANXA2 Deficiency Reveals a Maternal Cause of Preeclampsia

### Materials & Methods

Tissue Collection, hESC Isolation, and Culture

IRB approval was obtained on Aug. 8, 2011 by the CEIC Ethics Committee of Hospital La Fe, Valencia Spain (code 2011/0383) and informed written consent was signed from each patient prior to tissue collection. Severe Preeclampsia (sPE) endometrial biopsies (n=13) were obtained from women that have suffered sPE during their last pregnancy that occurred between 1 to 5 years. Non-Preeclampsia (Non-PE) endometrial biopsies were collected from women with normal pregnancies aged 18-32 years (n=13). All patients had regular menstrual cycles, with no underlying endometrial pathology and did not received hormonal treatment in the 3 months preceding biopsy collection. The mean age and mean BMI was similar in the two groups.

Endometrial biopsy was obtained using pipelle (Genetics, Belgium) under sterile conditions. Samples were processed and the stromal compartment isolated by mild collagenase digestion as previously described (41). Human endometrial stromal cell (hESC) cultures were grown using a medium composed of Dulbecco modified Eagle's medium (DMEM)/F12 (Sigma, Madrid, Spain) containing 10% charcoal stripped fetal bovine serum (FBS) and 0.1% antibiotics. hESCs for different assays were cultured in plates to confluence for 2 or 4 days.

### In Vitro Decidualization Protocols

Confluent hESC monolayers were decidualized with DMEM/F12 containing 2% FBS, 0.1% antibiotics and two different decidualization protocols: i) progesterone (P4) (1 μM) and β-estradiol (E2) (30 nM) during 9 days, renewing media every 3 days; ii) 8-bromo-cAMP (cAMP, Sigma) (0.5 mM) and Medroxy-Progesterone Acetate (MPA, Sigma) (1 μM) during 3 days. Control hESCs were cultured in parallel without inductors of the decidual reaction.

The characteristic decidual phenotype was confirmed biochemically by the analysis of PRL (Abnova) and IGFBP-1 (Raybiotech) protein levels in the conditioned culture media by ELISA, and morphologically by F-actin staining. hESCs were cultured in plastic plates to 30-40% of confluence. To minimize the effects of epitope masking, cells were fixed with low concentrations of fixative (2-3% paraformaldehyde) and blocked with 5% BSA. Cells were incubated with 0.1 μg/mL Phalloidin-tetramethylrhodamine B isothiocyanate conjugate from Amanita Phalloides (Sigma Aldrich, USA) to F-actin, for 30 min at room temperature in the dark. Fluorescence confocal images were obtained with a Nikon microscope equipped with a 100×1.45 numerical aperture objective and a Yokogawa spinning-disk confocal unit (PerkinElmer). For each immunofluorescence labeling, at least three different tissue preparations were used.

### Intra-and Extracellular ANXA2 Protein Assay

hESC cells were lysed in lysis buffer (50 mM Tris-HCl pH 8.0, 150 mM NaCl, 1% IGEPAL CA 360, 0.5% Na-DOC, 0.1% SDS and 0.5M EDTA). Protein extracts (25 μg/lane) were separated on a 10% SDS-PAGE gel, transferred to a polyvinylidene difluoride membrane (Hybond-P (hydrophobic polyvinylidenedifluoride membrane) (Amersham Biosciences, NJ, USA) by electrophoresis and blocked in PBS-buffered saline with 5% milk and 0.1% Tween. Membranes were incubated overnight at 4° C. with 1/2500 rabbit polyclonal anti-human Annexin II (Abcam, Cambridge, UK) and 1/2000 mouse monoclonal anti human β-actin (Santa Cruz, Calif., USA) and revealed with horseradish peroxidase-conjugated secondary goat anti-rabbit and goat anti-mouse IgG-HRP from Santa Cruz (Calif., USA) antibodies. Antibody-antigen complexes were detected using enhanced chemiluminescence ECL Plus reagent (Amersham Biosciences, CT, USA).

Protein extracts (2 μg/well) and conditioned media were analyzed by ELISA (R&D Systems, MN, USA). Inmobilized capture antibody specifically binds Annexin A2. After washing away un-bounded material, a biotinylated detection antibody specific for Annexin A2 is used to detect the bound Annexin A2, using a standard Streptavidin-HRP format. Three replicates were performed for each condition and the absorbance values were extrapolated to standard curve to establish human Annexin A2 concentration (pg/mL).

### ANXA2 Immunohistochemistry

Formalin-fixed and paraffin-embedded endometrial biopsies were sectioned and mounted on glass slides coated with Vectabond (Vector Laboratories, Burlingame, Calif., USA). After deparaffinization and rehydration, sections were rinsed 3 times with PBS for 5 min. Immunohistochemistry was performed on endometrial sections using the LSAB peroxidase kit (Dako, Carpinteria, Calif., USA). Nonspecific binding was blocked with 5% BSA in PBS. Sections were incubated for 1 h at room temperature with 1:100 rabbit polyclonal antihuman annexin II (Abcam, Cambridge, UK) diluted in PBS with 3% BSA. In the absence of antibodies, negative controls were incubated with PBS including 3% BSA. Secondary antibodies were included in the LSAB peroxidase kit (Dako), valid for rabbit origin primary antibodies. Staining was achieved with 3,30-diaminobenzidine (DAB) chromogen for a time of between 30 s and 1 min. After counterstaining with hematoxylin for 10 s and washing with distilled water, slides were mounted with entellan (Merck, Darmstadt, Germany).

ANXA2 siRNA

To silence ANXA2, a siRNA oligonucleotide with specificity for ANXA2 (CGGCCUGAGCGUCCAGAAATT, SEQ ID NO: 1) and negative control RNA duplexes was used, both with modification 3′-AlexaFluor488 (Qiagen CA, USA). hESCs were transfected with ANXA2 siRNA (100 nM) or siRNA negative control (100 nM). All the transfection experiments were performed using Lipofectamine 2000 (Invitrogen) and DMEM/F12 medium. Cells were incubated for 6 h at 37° C. with the treatment, and then the medium were renewed by fresh medium without siRNA.

### F-Actin/G-Actin In Vivo Assay

Free monomeric actin (G-actin) versus filamentous actin (F-actin) content in ESC cells subject to ANXA2 inhibition followed by decidual inductors (AMPc and MPA) was determined using the G-actin/F-actin in vivo assay kit (Cytoskeleton, CO, USA). Non-decidual control, control wild-type, ANXA2 siRNA and decidual ESC cells were homogenized in F-actin stabilization buffer at 37° C. Cell lysates were then cleared of unbroken cells with a low speed centrifuge (2000 rpm). Cleared lysates were then centrifuged at 100 000×g to separate soluble G-actin from insoluble F-actin. Fractions were then proportionally loaded on a polyacrylamide gel, separated by electrophoresis SDS-PAGE and transferred to a nitrocellulose membrane for probing with 1/500 anti-actin antibody (Cytoskeleton, CO, USA). Densitometric quantification of the western blot determined the G-actin ratio found in the cytosol versus the F-actin incorporated into the cytoskeleton normalized with total actin.

### Wound-Closure Assay

hESCs cells were seeded onto coverslips, grown to confluence and decidualized followed by ANXA2 siRNA treatment (6 h). After 96 h, each coverslip was scratched with a sterile pipette tip, washed with PBS and placed into fresh medium. The wound width was measured by phase-contrast microscopy immediately and after 24 h. Wound closure was calculated as a percentage of closed area of the initial wound width. The data shown represent the mean±SEM of ten measurements taken from three independent experiments.

### Trophoblast Spreading Assay

The protocol used was approved by the Animal Care and Use Committee of the Valencia University School of Medicine and in accordance with U.S. National Institutes of Health guidelines for the Care and Use of Laboratory Animals. The B6C3F1 mice strain was purchased from Charles River Laboratories (Barcelona, Spain). Female mice aged 6-8 weeks were superovulated and housed overnight in pairs with a stud male. On day 2 of pregnancy, embryos were recovered from the oviduct and cultured for 3 days in CCM-30 medium (Vitrolife, Lubeck, Germany). Only expanded blastocysts with normal morphology were included in the study (n=425 mouse embryos)

Hatched embryos were cocultured on confluent decidualized hESCs monolayer acting as controls, control siRNA or ANXA2 siRNA. After 48 h, the trophoblast spreading area of blastocyst attached was evaluated. Co-cultures were fixed with low concentrations of fixative (2-3% paraformaldehyde) and blocked with 5% BSA, incubated with primary antibodies include 1/50 mouse anti Vimentin (Sigma Aldrich, USA) and 1/100 rabbit anti E-cadherin (Abcam, Cambridge, UK) diluted in 3% BSA for 2 h at room temperature. Cells were incubated with secondary antibody 1/1000 TRICT anti-mouse (Invitrogen, Barcelona, spain) to vimentin and 1/1000 Alexa Fluor 488 anti-rabbit (Invitrogen, Barcelona, Spain) to E-cadherin, for 1 h at room temperature in the dark. 10-15 mouse blastocysts per condition were assessed in each experiment. The outgrowth area (expressed in pixels) was expressed as mean values±SEM of triplicate sets of measurements taken from three independent experiments.

### Invasion Assay

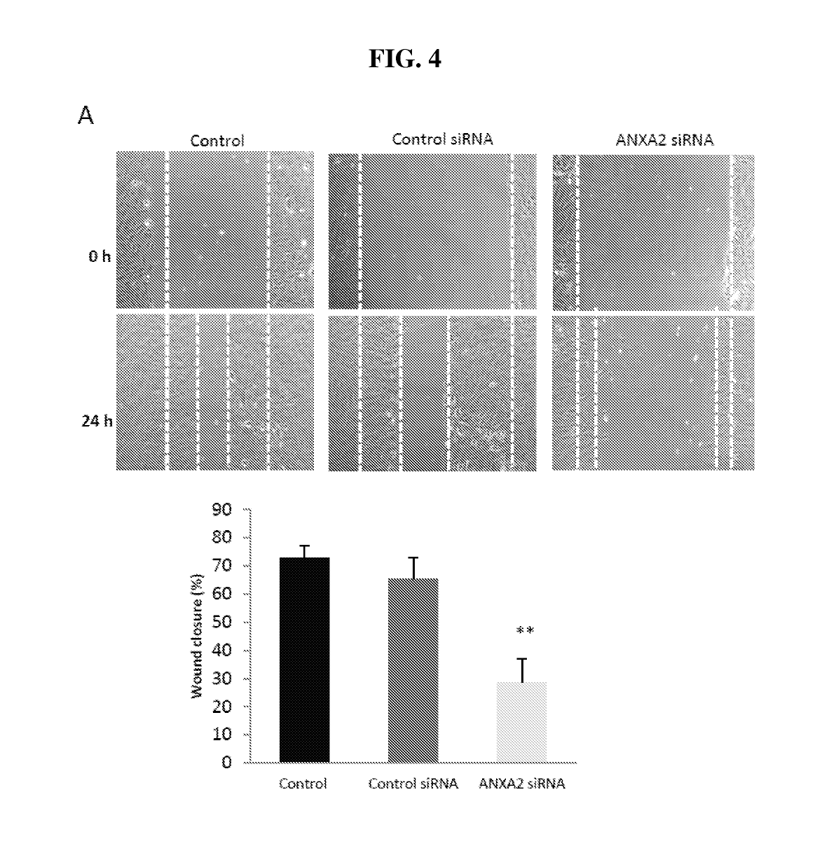

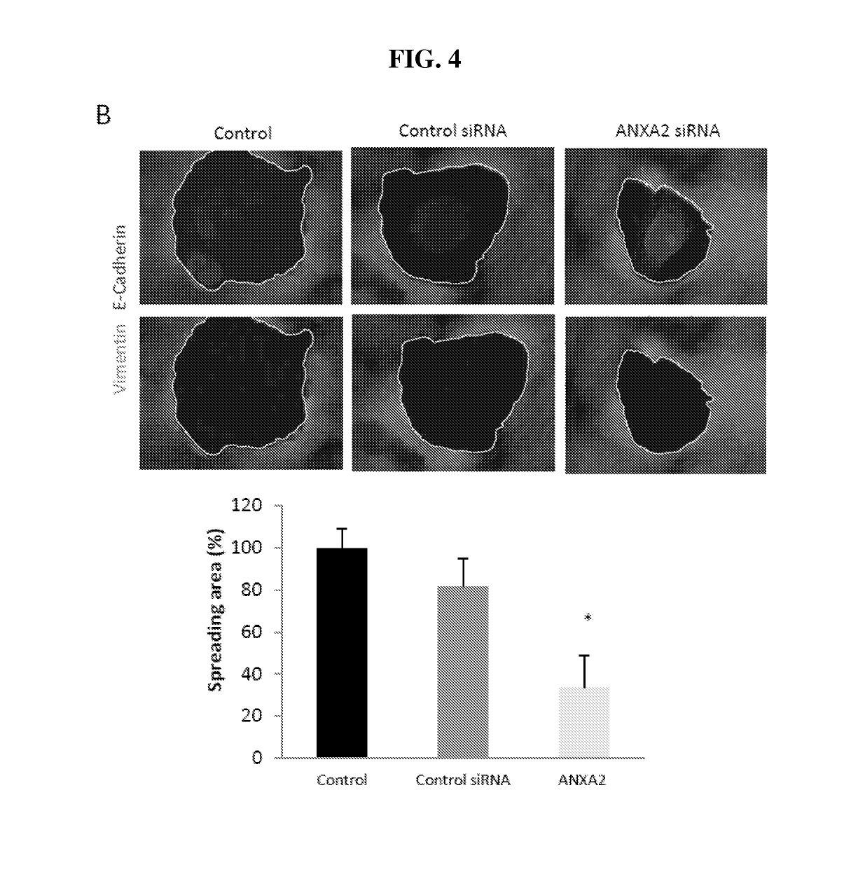

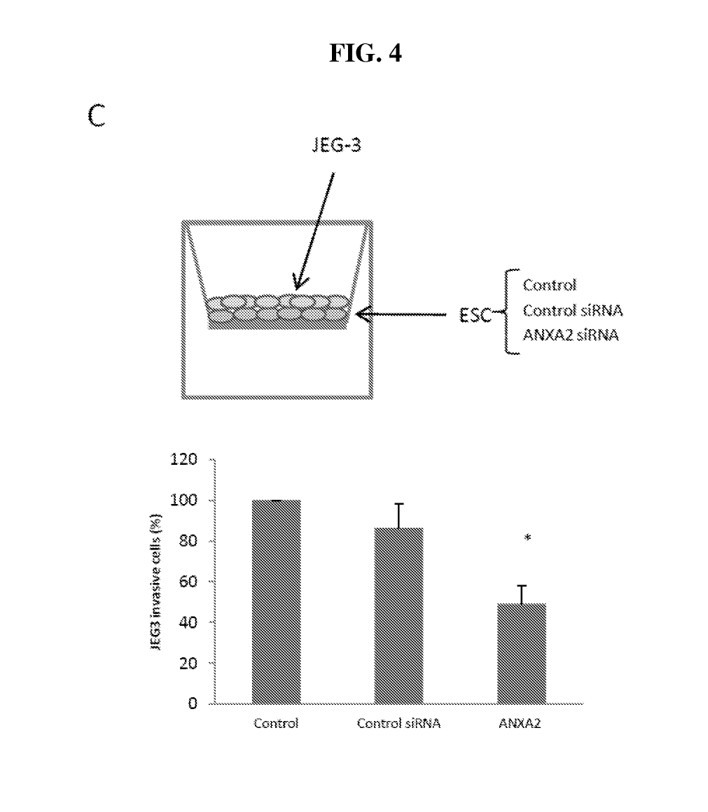

Trophoblast-derived cell line JEG-3 was used to evaluate the ability of trophoblast to invade through decidual hESC (Ref. Hannan 2010). Invasion assays were performed with the Collagen Transwell Invasion kits (Chemicon Int. Billerica, MA). 5×10⁵ decidualized hESC as controls, control siRNA or ANXA2 siRNA were grown to confluence into 8-mm-pore size transwell inserts during 24 h. On the top of insert, 10⁶ JEG-3 cells were resuspended in the hESCs medium and JEG-3 cells were allowed to invade for 48 h. Invasion was measured by OD using a standard microplate reader (FIG. 4C).

### Fibrinolysis Study

Plasminogen level was evaluated in conditioned media by ELISA kit (Cell Biolabs, CA, USA) following manufacturer's instructions. Average absorbance (Δ450 nm) in duplicate wells was calculated by subtracting the background from wells with media without hESC.

Plasmin activity was measured in conditioned media by fluorimetric assay kit (Anaspec, CA, USA). It is based in a protease cleavage to plasmin substrate that generates a rhodamine 110 fluorophore which has a bright green fluorescence detected at 496 nm/520 nm (excitation/emission). Fifty microlitres of conditioned media was pre-incubated 10 min at room temperature and 50 uL of plasmin substrate solution was added into each well. Fluorescence signal was obtain for kinetic reading immediately start measuring and record data every 5 min for 60 min, with a total of 13 lectures. Each condition was evaluated in duplicate. The fluorescence was Interpol to concentration values using an Rh110 fluorescence reference standard.

### MMP2 and MMP9 Levels

MMP2 and MMP9 pro and active protein forms were evaluated by commercial ELISA analysis (RayBiotech, GA, USA). These assays employ an antibody specific for humans MMP-2 and MMP-9 coated on a 96-well plate. Standards and samples are pipette by duplicated into the wells and MMP-2 and MMP-9 present in a sample is bound to the wells by the immobilized antibody. Biotinylated anti-human MMP-2 and MMP-9 and HRP-conjugated streptavidin were added. TMB substrate solutions were added and absorbance at 450 nm was extrapolated to standard curve.

### Quantitative PCR

Total RNA was extracted from hESC cultures using Trizol LS reagent (Invitrogen, Barcelona, Spain) according to the manufacturer's instructions. Firstly, 1 μg of total RNA was reverse-transcribed into cDNA using the Advantage RT-for-PCR kit (Clontech CA, USA) following the manufacturer's instructions. Quantitative real-time PCR was performed using SYBR Green (Roche) in a Light Cycler 480 system (Roche). Transcripts were quantified from the corresponding standard curve, using GAPDH as an internal control. Each experiment was performed three times with each sample in triplicate. The following primers were used:

ANXA2
(Fw: TGTGCAAGCTCAGCTTGGA, SEQ ID NO: 2,
Rv AGGTGTCTTCAATAGGCCCAA, SEQ ID NO: 3),
and
GAPDH
(Fw GAAGGTGAAGGTCGGAGTC, SEQ ID NO: 4,
Rv GAAGATGGTGATGGGATTTC, SEQ ID NO: 5).

### Heparin Dose-Response

hESCs were cultured on monolayer in presence of 50 μg/mL and 100 μg/mL of heparin (Sigma, Madrid, during 15, 30 and 60 min to elaborate a dose-response experiment. Conditioned media from hESC cells were collected to analyze plasminogen levels, plasmin activity and metalloproteases production.

### Statistical Analysis

At least three different endometrial biopsies were used per each experiment and measurements were taken in triplicate. Mean values±SEM are presented with n denoting the number of experiments. Data were analyzed with SPSS software using the t-test for the analyzed global differences between groups. A p-value of P≦0.05 was considered significant. (*p≦0.05, **p≦0.01, ***p≦0.001)

### Results

In Vitro Decidualization Resistance In Patients that Suffered sPE In Their Previous Pregnancies

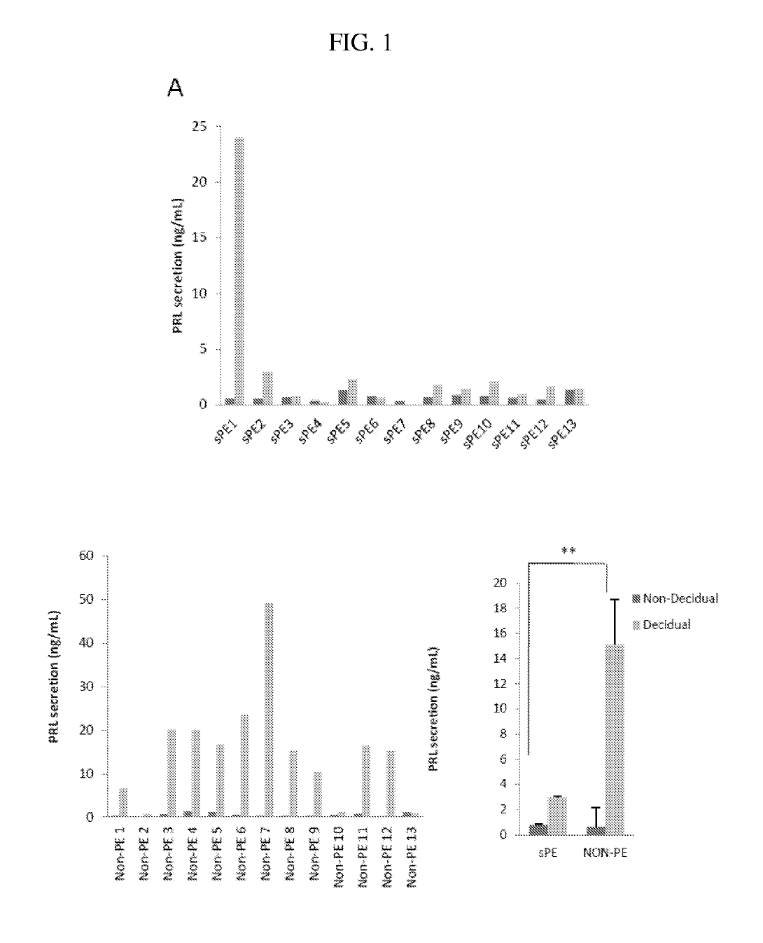

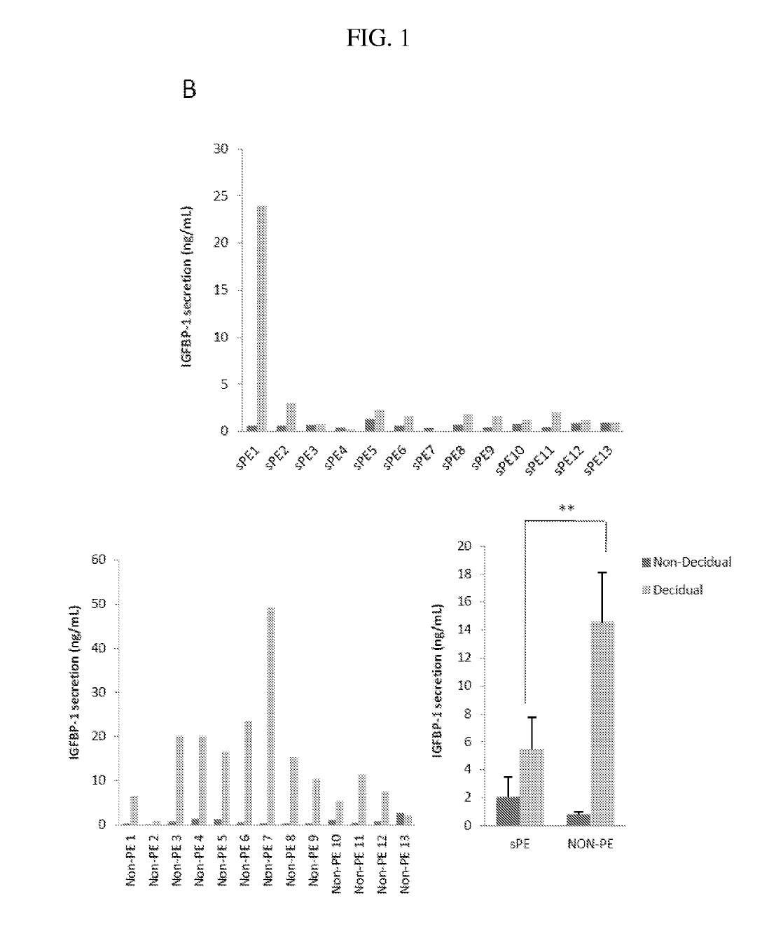

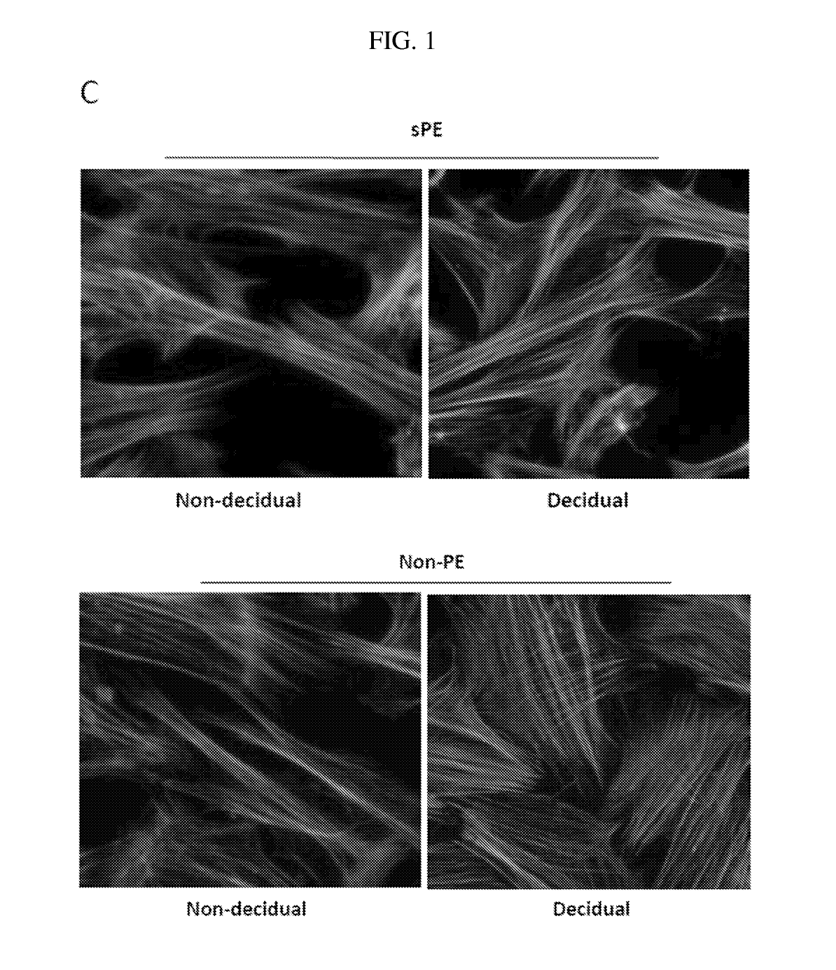

In vitro decidualization of hESCs from women that have suffered severe PE (sPE) in their previous pregnancies (n=13) compared to control patients with history of normal pregnancies (non-PE) (n=13) was assessed. In the sPE group, hESCs were isolated from women who suffered different forms including superimposed HELLP syndrome, eclampsia, or two consecutive previous sPE developing HELLP syndrome that ended in eclampsia. The patients with sPE and non-PE had comparable BMIs and ages, but women with sPE had higher systolic/diastolic blood pressures, proteinuria, GOT, GPT and lower platelets count and fibrinogen levels. hESCs decidualized either with cAMP (0.5 μM)+MPA (1 μM) for five days as decidual stimulus or with hormonal inductors P4 (1 μM)+E2 (30 nM) for 9 days showed similar results. Therefore the cAMP+MPA protocol was used for this study. Interestingly, PRL and IGFBP-1 secretion demonstrate that in vitro decidualization was impaired in hESC obtained from sPE compared to the non-PE counterparts (FIGS. 1A and 1B, respectively). F-actin reorganization in hESCs was investigated during in vitro decidualization and showed a transition from fibroblastic phenotype to enlarged rounded cell morphology in non-PE, whereas the transition to decidual phenotype was absent in decidualized hESC from sPE patients (FIG. 1C).

Downregulated and Deregulated hESC Expression of ANXA2 In sPE

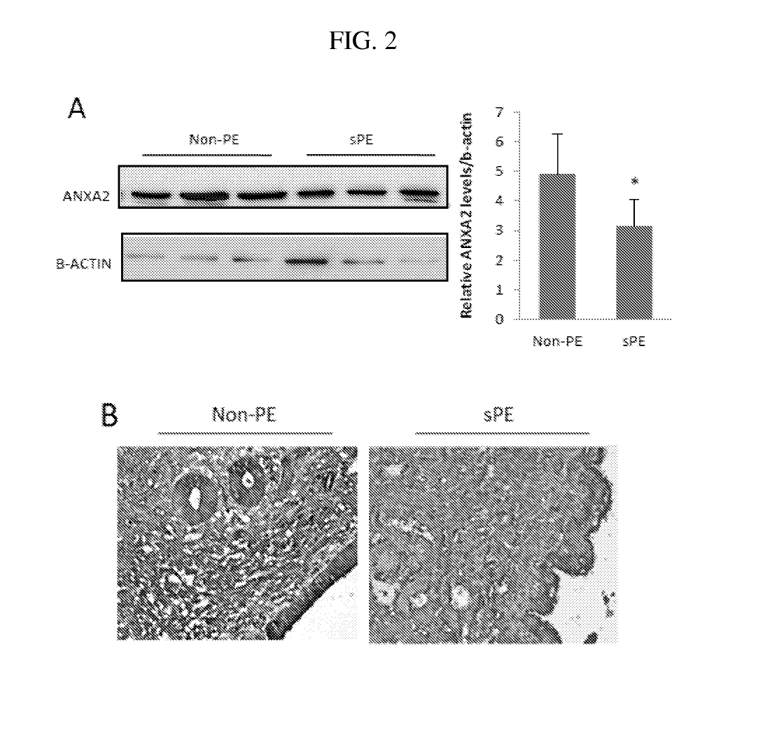

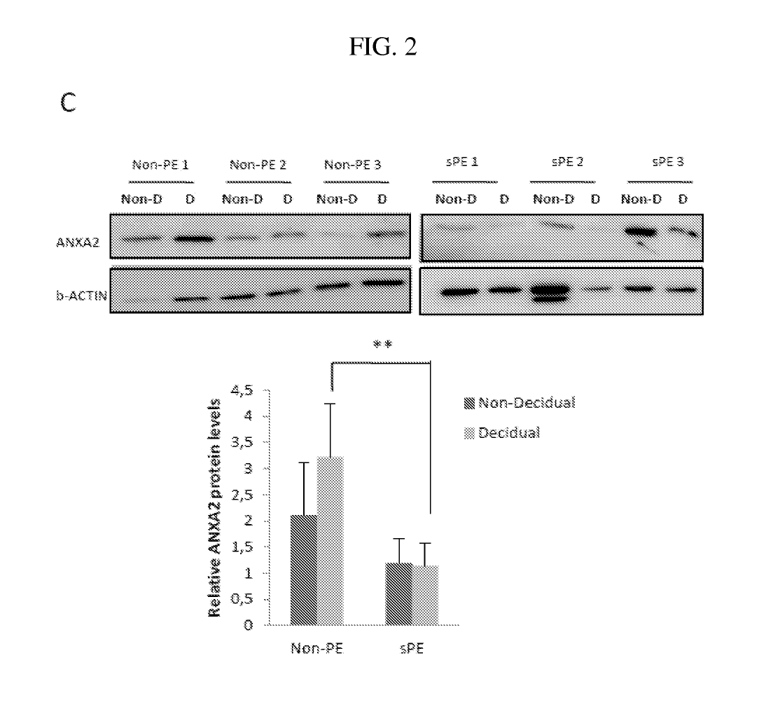

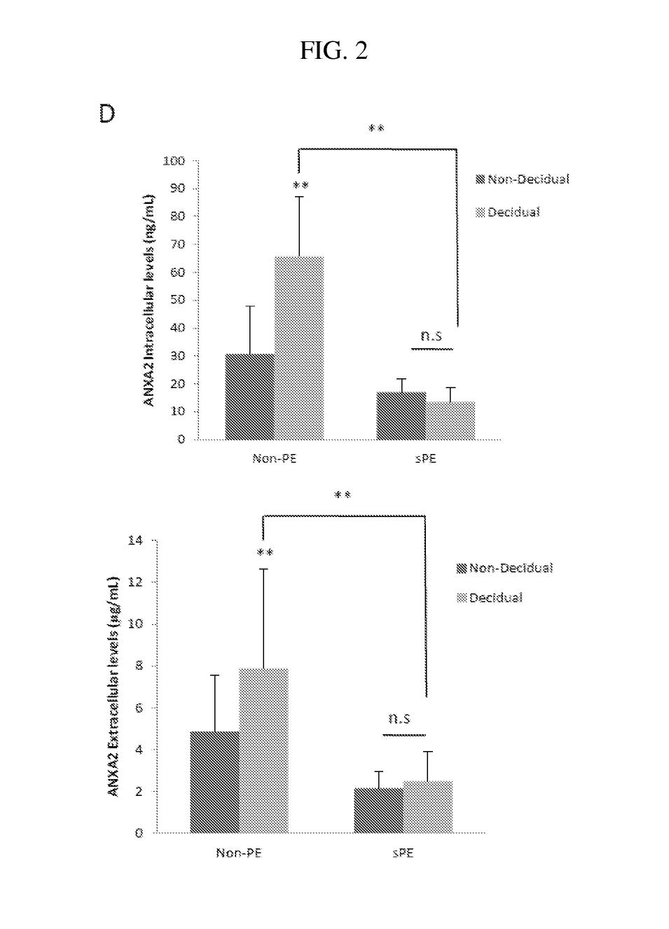

ANXA2 protein abundance in whole endometrial samples from women that suffered sPE in their previous pregnancy vs non-PE patients was analyzed (FIG. 2A). Densitometric analysis showed significant reduction in ANXA2 abundance in the endometrium from sPE (n=6) compared to non-PE patients (n=6) (FIG. 2A). The endometrial ANXA2 localization was examined. A lower staining at the stromal compartment in sPE versus non-PE was observed (FIG. 2B). Next, hESCs from sPE versus non-PE patients were isolated, decidualized and ANXA2 protein were assessed by western blot (FIG. 2C). Densitometric analysis demonstrated that in sPE women, ANXA2 was significantly reduced in the basal conditions and deregulated in decidualized hESC compared to non-PE patients (FIG. 2C). To further quantify this molecule, intracellular and secreted ANXA2 forms during decidualization in both conditions were analyzed using ELISA. This analysis corroborates that hESCs from sPE women in the presence of decidualization stimulus experience a significant reduction and deregulation in both intracellular and extracellular ANXA2 compared to controls (FIG. 2D). Furthermore, the secreted form mirrors intracellular ANXA2, which confirms the use of ANXA2 as a biomarker that can be used to predict decidualization resistance in PE patients.

Regulation and Functionality of ANXA2 In hESCs During In Vitro Decidualization

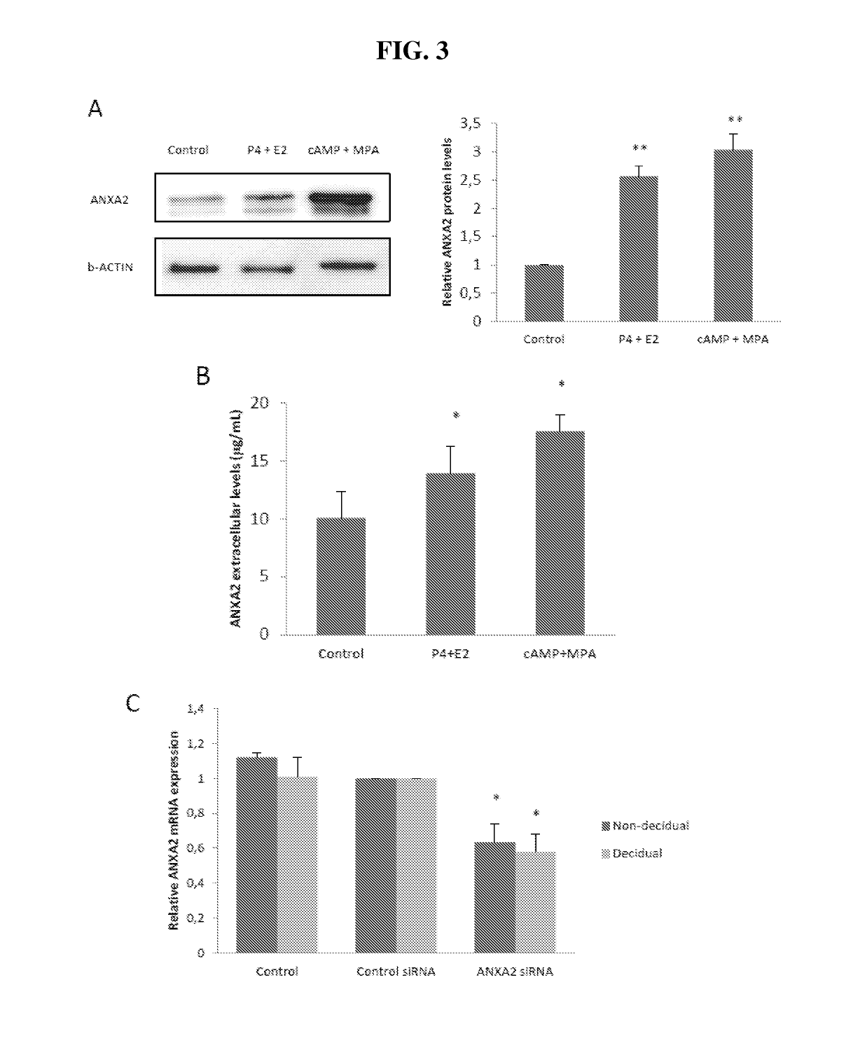

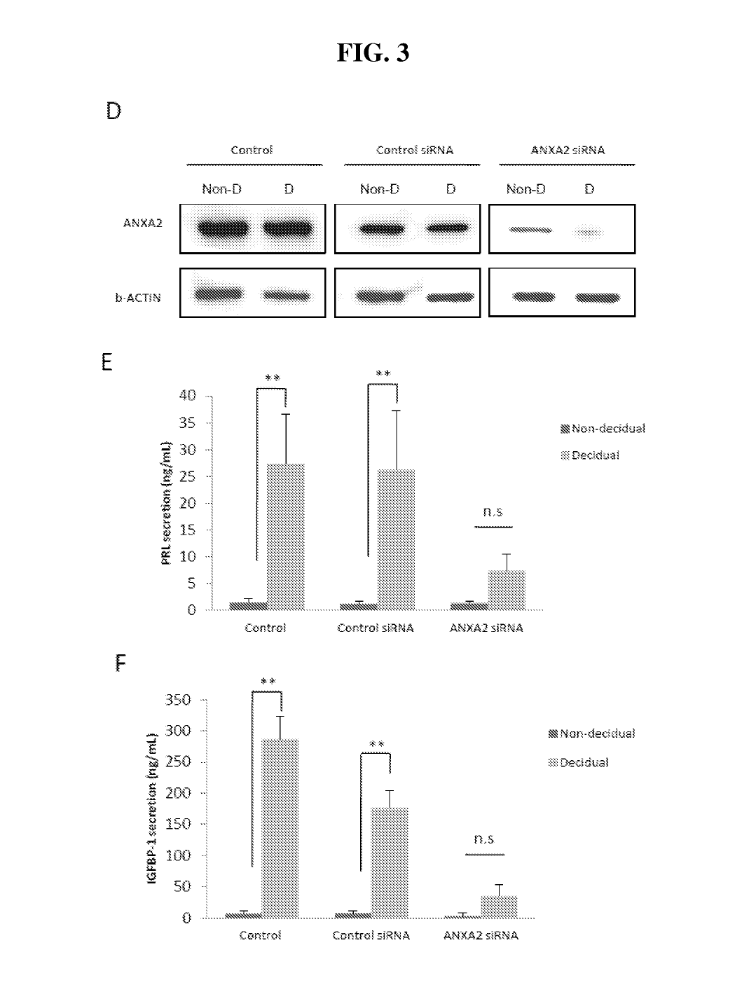

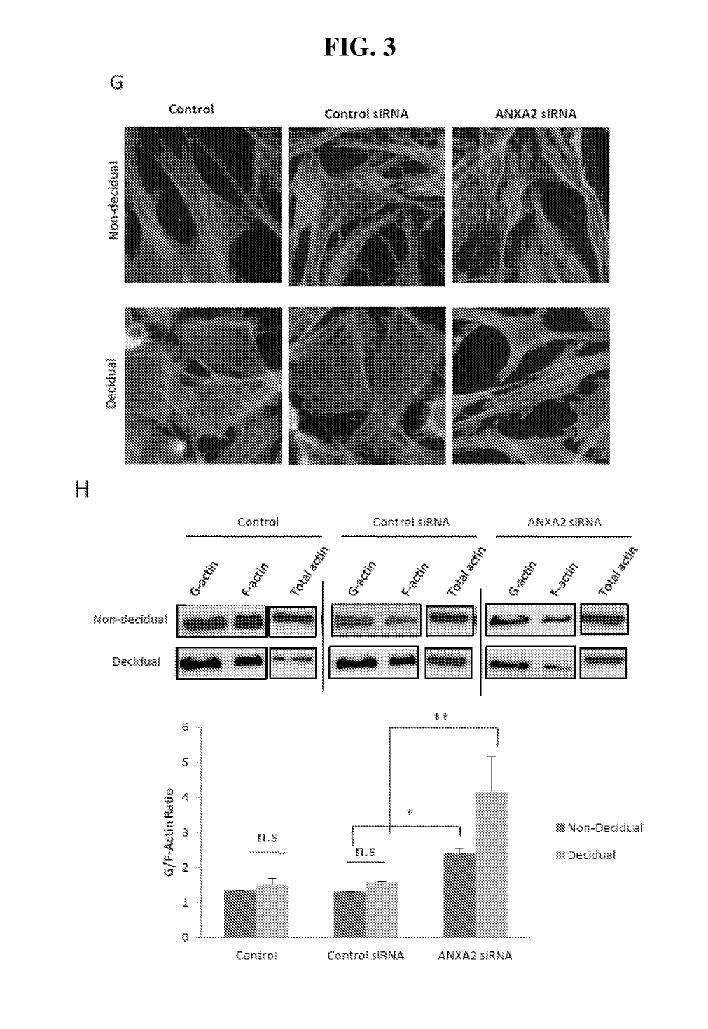

Previous studies show that ANXA2 is regulated throughout the menstrual cycle in human endometrium during receptivity acquisition and initial steps of embryo implantation. The regulation of ANXA2 during hESC decidualization in vitro was investigated next. Intracellular ANXA2 assessed by western blot (FIG. 3A) and densitometric analysis corroborated the upregulation of intracellular ANXA2 in decidualized vs non-decidualized hESCs using both protocols (FIG. 3A). Secreted ANXA2 was measured in the supernatant of hESCs by ELISA paralleling its intracellular dynamic (FIG. 3B). These results demonstrate that intra- and extracellular ANXA2 are upregulated in hESC during in vitro decidualization.

Next, the functionality of ANXA2 in hESCs was assessed during in vitro decidualization by inhibiting the ANXA2 molecule using a siRNA approach. Twenty-four hours after hESC transfection a significant reduction in ANXA2 mRNA (FIG. 3C) and protein (FIG. 4D) was observed in the siRNA group compared to the control and control siRNA groups in both non-decidual and decidual conditions. To corroborate its functional relevance, the impact of ANXA2 inhibition was evaluated in the secretion of decidual biomarkers such as PRL and IGFBP-1 as well as the morphological phenotypic changes at 72 h after initiation of the decidual stimulus. Unlike controls, PRL and IGFBP-1 were absent in siRNA decidualized hESCs (FIGS. 3E and 3F). Also, rhodamine-phalloidin staining confirmed that ANXA2 interference abrogated the characteristic phenotypic modifications of F-actin architecture during the decidualization process leaving the longitudinal orientation of the F-actin filaments unaltered (FIG. 3G). The reorganization of the actin cytoskeleton during the decidualization after ANXA2 inhibition was also investigated using the ratio of the free monomeric G-actin found in the cytosol compared to the F-actin incorporated into the cytoskeleton (FIG. 3H). The average G/F-actin ratio was approximately 1:1 in control and control siRNA hESC cells in both decidual and non-decidual phenotype, whereas the ANXA2-inhibited hESC cells revealed a significant increase in the content of monomeric G-actin compared to F-actin (a ratio of 3:1 in the non-decidual and of 4:1 in decidual ANXA2 siRNA-treated cells) (FIG. 3H). These data demonstrate that ANXA2 inhibition induces a decidualization resistance through actin filament depolymerization and a significant increase in the G-actin monomeric fraction, demonstrating a functional role of ANXA2 in the reorganization of F-actin fibers during the decidualization process.

ANXA2 Inhibition Reduces hESC Motility, Trophoblast Spreading and Invasion

To further understand the paracrine actions of the decidualization resistance induced by ANXA2 inhibition on trophoblast spreading and invasion, a wound-closure assay was performed to analyze the implication of ANXA2 in hESCs motility. Decidualization of hESCs was followed by the transfection with ANXA2 siRNA for 6 h, then the monolayer of cells was disrupted with a scratch, and the effects of ANXA2 inhibition in terms of migration, were tracked during 24 hours by video microscopy (FIG. 4A). Percentage of wound closure in the ANXA2 siRNA-inhibited was significantly reduced compared to the control and control siRNA cells (FIG. 4A).

The effect of ANXA2 inhibition on trophoblast spreading, using a heterologous in vitro co-culture model where mouse embryos were placed onto a confluent decidualized hESC monolayer followed by ANXA2 siRNA inhibition was studied next. Immunostaining for E-cadherin and vimentin identify mouse trophoblast and hESCs, respectively. Total area of trophoblast spreading was evaluated as a number of pixels and a significant decrease in ANXA2 siRNA compared to control and control siRNA hESC cells was observed (FIG. 4B).

The invasiveness of JEG-3 human trophoblast cell line into ANXA2 inhibited hESC cells was also analysed using a collagen-invasion chamber assay. Decidualized hESCs were ANXA2 siRNA inhibited and cultured in inserts on a collagen layer. Then, a JEG-3 cell suspension was placed on top of inserts. The ability to invade through the monolayer of treated hESCs and the collagen barrier was examined. The percentages of invading JEG-3 cells in ANXA2 inhibited cells were significantly reduced compared to control hESC (FIG. 4C).

Deficient Fibrinolytic Activity Due to Inhibition of ANXA2 is Also Present In hESC from sPE

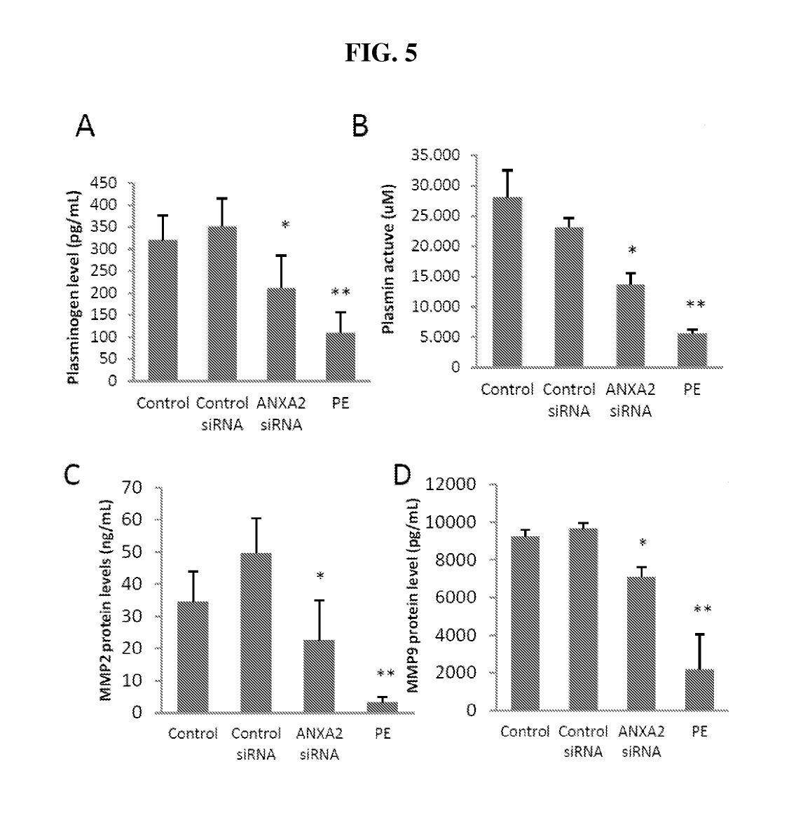

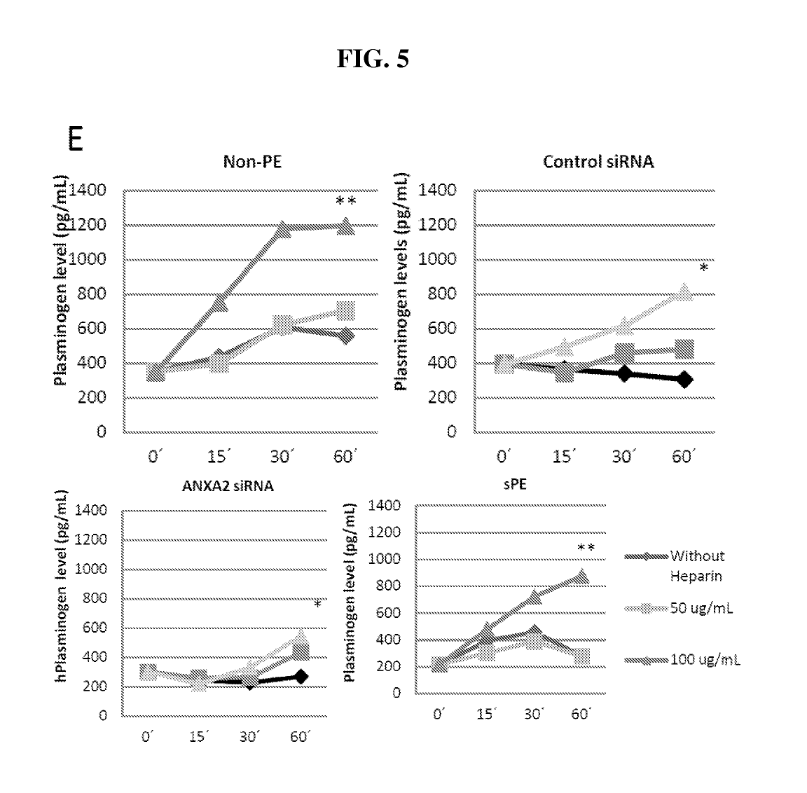

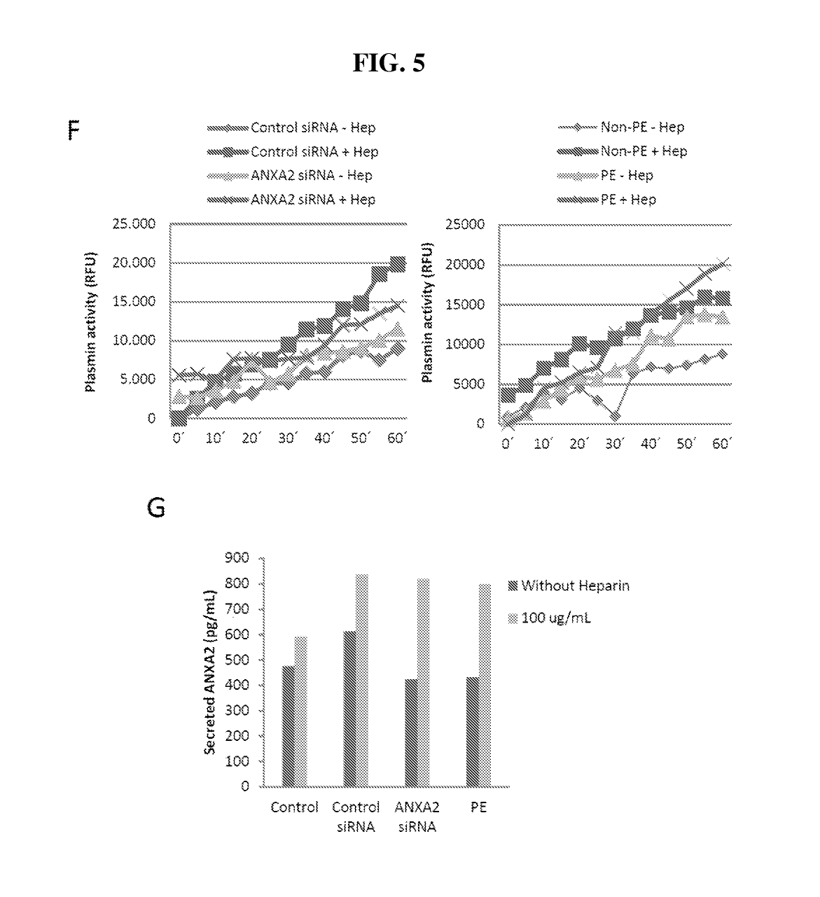

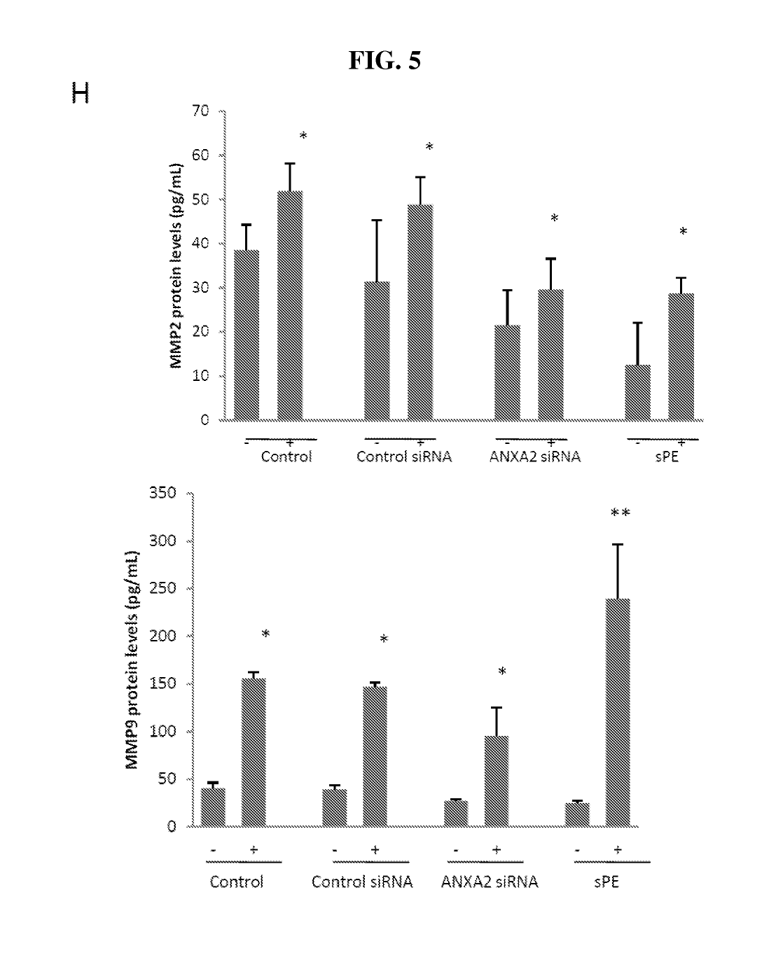

The fibrinolytic system is implicated in the pathogenesis of PE through fibrin deposition and a predisposition to endothelial dysfunction. The functional effect of hESC ANXA2 inhibition on fibrinolytic activity compared to hESC from PE women was investigated. For this purpose, plasminogen levels, and plasmin activity in conditioned media from decidualized controls, ANXA2 siRNA and hESCs from PE were analyzed. Plasminogen levels and plasmin activity were significantly reduced in ANXA2 siRNA and hESCs from sPE compared to control siRNA hESCs and control decidualized hESCs (plasminogen levels: 229.1±23.1 and 191.5±36.7 pg/mL versus 305.1±23.2 and 397.1±45.1 pg/mL, respectively; plasmin activity: 12.7±3.6 mM and 4.2±0.75 mM versus 27.5±10.2 mM and 23.1±4.1, respectively) (FIGS. 5A and 5B). Therefore, the fibrinolysis system is deficient when ANXA2 is inhibited in decidualized hESC and to a greater extent in hESC from SPE patients.

Interestingly the plasminogen/plasmin system regulates trophoblast invasion by the production of MMP2 and MMP9 proteins that degrade ECM components such as fibrin and collagen. MMP2 and MMP9 protein secretion was analyzed in conditioned media of ANXA2 inhibited and sPE decidualized hESCs by ELISA. Levels of MMP2 and MMP9 secretion were significantly reduced when ANXA2 was inhibited and in sPE patients (FIGS. 5C and 5D).

Heparin Treatment Favors the Activation of Defective Fibrinolysis System In ANXA2 Reduced hESC

Heparin acts on the fibrinolysis pathway through tissue plasminogen activator (tPA), and also has been described as a direct effect of heparin binding to ANXA2. The effect of heparin on fibrinolysis was analyzed in dose-response and time-dependent experiments measuring plasminogen abundance and plasmin activity on control siRNA, ANXA2 siRNA inhibited, non-PE and PE decidualized hESCs. Heparin at 100 ug/mL significantly increased plasminogen and plasmin secretion into the conditioned media in all conditions investigated, including in ANXA2 inhibited and sPE decidualized hESC (FIG. 5E). Also, heparin effect on plasmin was also evaluated, resulting in a significant increase of plasmin activity on ANXA2 siRNA and hESCs from sPE at the same dose (FIG. 5F). Secreted extracellular ANXA2 levels in conditioned media of control, ANXA2 siRNA and hESC from sPE patients were measured by ELISA. The results corroborated that heparin treatment induce a significant increase in ANXA2 protein secreted to the culture media (FIG. 5G).

Finally, the functional effect of heparin on MMP2 and MMP9 metalloproteases production in all conditions was tested in vitro (FIG. 5H). Treatment with heparin induced a significant increase of metalloproteases, key elements to facilitate trophoblast invasion through endometrial stromal cells. Therefore, direct and/or indirect effects of heparin on ANXA2 deficient decidualized hESC either induced by siRNA or naturally occurring in patients with sPE correct at least in part, the associated fibrinolytic defect.

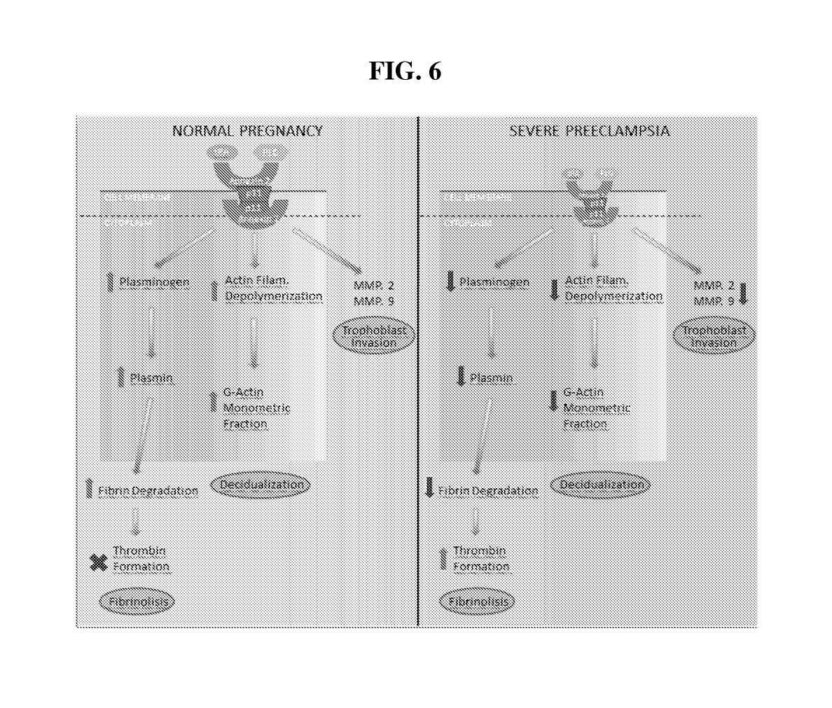

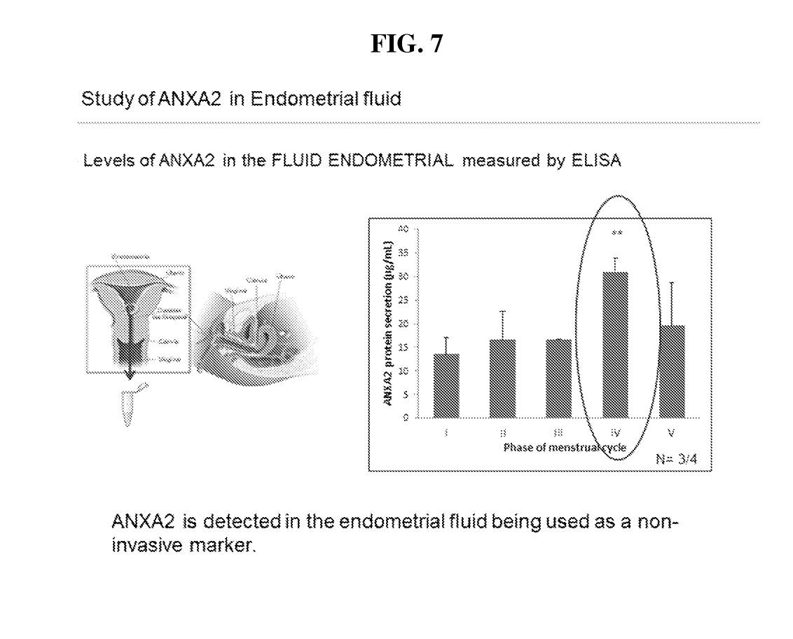

Based on these data, a model is proposed that integrates hESC decidualization resistance present in sPE, mediated at least in part by ANXA2 deficiency, with shallow trophoblast invasion and fibrinolytic alterations as a maternal cause of PE (FIG. 7). It was found that ANXA2 deficient hESC in sPE or induced trough siRNA do not decidualize properly due to actin filaments depolymerization and a significant increase in the G-actin monomeric fraction impeding their typical morphological transformation. A major downstream consequence included direct effect on the pro-enzyme plasminogen leading to reduce plasmin generation creating a protrombotic paracrine effect due a deficiency in the fibrinolysis system. Similarly ANXA2 activation deficiency leads to shallow trophoblast invasion trough the inhibition of MMP2 and MMP9 that degrade ECM components such as fibrin and collagen. Addition of heparin acting through ANXA2 is able to overcome the indicated downstream effects.

### Discussion

Although defective CTB differentiation as a possible cause of PE is under intense investigation, this work focused on the endometrial maternal paracrine factors involved in the origin of this obstetric complication. Epidemiological studies reveal that previous PE in the maternal family is associated with a 24%-163% increased risk to suffer PE in female relatives. However, PE episodes in the paternal family do not affect PE risk in a given patient. Thus, the genetic susceptibility for PE is clearly associated with the maternal lines.

hESCs were targeted through the decidualization transformation from the decidua that regulates the invasion of the CTB in the uterine wall. Identification of decidualization resistance in hESCs obtained from sPE compared to non-PE counterparts prompted the study.

Annexin A2 (ANXA2) is a calcium-regulated phospholipid binding protein that is significantly up-regulated during the mid- and late-secretory phases of the human endometrium. This protein is key to the acquisition of the receptive phenotype by the endometrial epithelium by the modulation the F-actin network. ANXA2 is a profibrinolytic receptor, present and functional on hESC. It acts as a cell surface co-receptor for plasminogen and its activator tPA, significantly enhanced cell surface plasmin generation. Due to the alteration of the fibrinolytic pathway in PE, the mechanistic analyses were focused on ANXA2 because high titers of this molecule have been associated with thrombotic events in antiphospholipid syndrome (APS), a condition known to predispose the development of PE. Moreover, ANXA2 autoantibodies in placentas with PE have been suggested as a possible cause of placental thrombin formation.

Initially, ANXA2 in the endometrial stromal compartment was reduced in patients that suffer sPE in their previous pregnancies vs non-PE. Next, the analysis corroborated that hESC from sPE women in the presence of decidualization stimulus experience a significant reduction and deregulation in both intracellular and extracellular ANXA2 compared to controls. Another surprising finding was that intra- and extracellular ANXA2 were upregulated in hESC during in vitro decidualization and its functional inhibition induced decidualization resistance through actin filaments depolymerization and increase of the G-actin monomeric fraction. Further investigation of the autocrine and paracrine actions of the decidualization resistance induced by ANXA2 inhibition revealed a direct effect on hESC motility, and reduction of trophoblast spreading and invasion which are hallmarks of this pathological condition.

Fibrinolysis is a well-organized process through which the plasminogen is converted to plasmin, through the action of tissue plasminogen activator (tPA) and urokinase plasminogen activator (uPA), that remodel and degrade fibrin thrombi. A common histopathological finding in placentas from PE is the appearance of various degrees of thrombosis together with fibrin deposits. Defects of fibrinolytic function are known risk factors for increased thrombosis. Alterations of fibrinolysis are present in PE, suggesting a fibrinolytic abnormality in the development of the disease, either as cause or consequence. ANXA2 has a direct effect on the pro-enzyme plasminogen and to the same extrinsic fibrinolysis pathway, therefore the fibrinolysis system was deficient in decidualized hESC when ANXA2 was inhibited, and to a greater extent in hESC from sPE patients with decidualization resistance. The alteration of the plasminogen/plasmin system impaired trophoblast invasion trough the inhibition of MMP2 and MMP9 that degrade ECM components such as fibrin and collagen. It was also demonstrated that heparin at a dose of 100 ug/mL in vitro increased secreted ANXA2 protein, plasminogen, plasmin, MMP2 and MMP9 production in all conditions investigated. Therefore, this research settles the fundaments to understand the beneficial effect reported of heparin treatment in PE.

### REFERENCES

1. Steegers E A, von Dadelszen P, duvekot J J, Pijnenborg R. Pre-eclampsia. Lancet 2010; 376: 631-644.
2. Gifford R W, August P A, Cunningham G, Green L A, Lindheimer M D, McNellis D, AL E. Report of the National High Blood Pressure Education Program Working Group on High Blood Pressure in Pregnancy. Am J Obstet Gynecol. 2000; 183, S1-S22.
3. Roberts, J. M. & Gammill, H. S. Preeclampsia: recent insights. Hypertension 2005; 46, 1243-1249.
4. Pijnenborg, R., Vercruysse, L. & Hanssens, M. Fetal-maternal conflict, trophoblast invasion, preeclampsia, and the red queen. Hypertens Pregnancy 2008; 27, 183-196.
5. Roberts D J, Post M D. The placenta in preeclampsia and intrauterine growth restriction. J Clin Pathol. 2008; 61: 1254-60.
6. Roberts J M, Taylor R N, Goldfien A. Clinical and biochemical evidence of endothelial cell dysfunction in the pregnancy syndrome preeclampsia. Am J Hypertens 1991; 4, 700-708.
7. Vinnars M T, Nasiell J, Ghazi S, Westgren M, Papadogiannakis N. The severity of clinical manifestations in preeclampsia correlates with the amount of placental infarction. Acta Obstet Gynecol Scand. 2011; 90: 19-25.
8. Naicker T, Khedun S M, Moodley J, Pijnenborg R. Quantitative analysis of trophoblast invasion in preeclampsia. Acta Obstet Gynecol Scand. 2003; 82: 722-729.
9. Brosens I A, Robertson W B, Dixon H G. The role of the spiral arteries in the pathogenesis of preeclampsia. Obstet Gynecol Annu. 1972; 1: 177-191.
10. Zhou Y, Damsky C H, Chiu K, Roberts J M, Fisher S J. Preeclampsia is associated with abnormal expression of adhesion molecules by invasive cytotrophoblasts. J Clin Invest. 1993; 91: 950-960.
11. Zhou Y, Damsky C H, Fisher S J. Preeclampsia is associated with failure of human cytotrophoblasts to mimic a vascular adhesion phenotype. One cause of defective endovascular invasion in this syndrome? J Clin Invest. 1997; 99: 2152-2164.
12. Zhou Y, Gormley M J, Hunkapiller N M, Kapidzic M, Stolyarov Y, Feng V, Nishida M, Drake P M, Bianco K, Wang F, McMaster M T, Fisher S J. Reversal of gene dysregulation in cultured cytotrophoblasts reveals possible causes of preeclampsia. J Clin Invest. 2013; 123: 2862-2872.
13. Irwin J C, Utian W H, Eckert R L. Sex steroids and growth factors differentially regulate the growth and differentiation of cultured human endometrial stromal cells. Endocrinology 1991; 129: 2385-2392.
14. Brar A K, Frank G R, Kessler C A, Cedars M I, Handwerger S. Progesterone-dependent decidualization of the human endometrium is mediated by cAMP. Endocrine 1997; 6: 301-307.
15. Gellersen B, Reimann K, Samalecos A, Aupers S, Bamberger A M. Invasion of human endometrial stromal cells is promoted by decidualization and by trophoblast-derived signals. Hum Reprod 2010; 25: 862-873.
16. Dunn C L, Kelly R W, Critchley H O 2003 Decidualization of the human endometrial stromal cell: an enigmatic transformation. Reprod Biomed Online 7: 151-161
17. Giudice L C, Mark S P, Irwin J C. Paracrine actions of insulin-like growth factors and IGF binding protein-1 in non-pregnant human endometrium and at the decidual-trophoblast interface. J Reprod Immunol. 1998; 39: 133-48.
18. Jabbour H N, Critchley H O. Potential roles of decidual prolactin in early pregnancy. Reproduction. 2001; 121: 197-205.
19. Ramathal C Y, Bagchi I C, Taylor R N, Bagchi M K. Endometrial decidualization of mice and men. Semen Reprod Med. 2010; 28: 17-26.
20. Tabanelli S, Tang B, Gurpide E. In vitro decidualization of human endometrial stromal cells. J Steroid Biochem Mol Biol 1992; 42: 337-44
21. Garrido-Gomez T, Dominguez F, Lopez J A, Camafeita E, Quiñonero A, Martinez-Conejero J A, Pellicer A, Conesa A, Simón C. Modeling human endometrial decidualization from the interaction between proteome and secretome. J Clin Endocrinol Metab. 2011; 96: 706-716.
22. Garrido-Gómez T, Dominguez F, Quiñonero A, Estella C, Vilella F, Pellicer A, Simon C. Annexin A2 is critical for embryo adhesiveness to the human endometrium by RhoA activation through F-actin regulation. FASEB J. 2012; 26: 3715-3727.
23. Cesarman-Maus G, Ríos-Luna N P, Deora A B, Huang B, Villa R, Cravioto M del C, et al. Autoantibodies against the fibrinolytic receptor, annexin 2, in antiphospholipid syndrome. Blood. 2006; 107(11): 4375-4382.
24. Chu S C, Yang S F, Lue K H, Hsieh Y S, Hsiao T Y, Lu K H. Urokinase-type plasminogen activator, receptor, and inhibitor correlating with gelatinase-B (MMP-9) contribute to inflammation in gouty arthritis of the knee. J Rheumatol. 2006; 33(2): 311-317.
25. Beier J I, Kaiser J P, Guo L, Martínez-Maldonado M, Arteel G E. Plasminogen activator inhibitor-1 deficient mice are protected from angiotensin II-induced fibrosis. Arch Biochem Biophys. 2011; 510(1): 19-26.
26. Kruse-Blinkenberg H O, Gormsen J. The influence of low dose heparin in elective surgery on blood coagulation, fibrinolysis, platelet function, antithrombin III and antiplasmin. Acta Chir Scand. 1980; 146: 375-382.
27. Pogliani E M, Vigo A, Cofrancesco E, Colombi M, Cristoforetti G, Marchetti G, Vercesi G, Radaelli F. Low-dose heparin in thoracic surgery: effect on blood coagulation and fibrinolysis system. Thromb Res. 1982; 27: 211-219.
28. Arnesen H, Engebretsen L F, Ugland O M, Seljeflot I, Kierulf P. Increased fibrinolytic activity after surgery induced by low dose heparin. Thromb Res. 1987; 45: 553-559.
29. Shao C, Zhang F, Kemp M M, Linhardt R J, Waisman D M, Head J F, Seaton B A. J Biol Chem. Crystallographic analysis of calcium-dependent heparin binding to annexin A2. 2006; 281: 31689-31695.
30. Boyd H A, Tahir H, Wohlfahrt J, Melbye M. Associations of personal and family preeclampsia history with the risk of early-, intermediate- and late-onset preeclampsia. Am J Epidemiol. 2013; 178: 1611-1619.
31. Domínguez F, Garrido-Gómez T, López J A, Camafeita E, Quiñonero A, Pellicer A, Simón C. Proteomic analysis of the human receptive versus non-receptive endometrium using differential in-gel electrophoresis and MALDI-MS unveils stathmin 1 and annexin A2 as differentially regulated. Hum Reprod. 2009; 24: 2607-2617.
32. Bharadwaj A, Bydoun M, Waisman 2013 Annexin A2 Heterotetramer: structure and function. Int J Mol Sci 2013
33. Cesarman G M, Guevara C A, Hajjar K A. An endothelial cell receptor for plasminogen/tissue plasminogen activator (t-PA). II. Annexin II-mediated enhancement of t-PA-dependent plasminogen activation. J Biol Chem. 1994; 269: 21198-211203.
34. LingQ, Jacovina A T, Deora A, FebbraioM, Simantov R, Silverstein R L. Annexin II regulates fibrin homeostasis and neoangiogenesis in vivo. J Clin Invest. 2004; 113(1): 38-48.
35. Cesarman-Maus G, Hajjar K A. Molecular mechanisms of fibrinolysis. Br J Haematol 2005; 129: 307-321.
36. Crabbe S J, Cloninger C C. Tissue plasminogen activator: a new thrombolytic agent. Clin Pharm. 1987; 6: 373-386.
37. Sobel B E. Fibrinolysis and activators of plasminogen. Heart Lung. 1987; 16: 775-779.
38. Gohil R, Peck G, Sharma P. The genetics of venous thromboembolism. A metaanalysis involving approximately 120 000 cases and 180 000 controlls. Thromb Haemost. 2009; 102: 360-370.
39. Sucak G T, Acar K, Sucak A, Kirazli S, Haznedar R. Increased global fibrinolytic capacity as a clue for activated fibrinolysis in pre-eclampsia. Blood Coagul Fibrinolysis. 2006; 17: 347-352.
40. Conserva V, Muggiasca M, Arrigoni L, Mantegazza V, Rossi E, Ferrazzi E. Recurrence and severity of abnormal pregnancy outcome in patients treated by low-molecular-weight heparin: a prospective pilot study. J Matern Fetal Neonatal Med. 2012; 25: 1467-1473.
41. Simón C, Mercader A, Garcia-Velasco J, Nikas G, Moreno C, Remohí J, Pellicer A. Coculture of human embryos with autologous human endometrial epithelial cells in patients with implantation failure. J Clin Endocrinol Metab. 1999; 84: 2638-2646.
42. Tackels-Horne, D, Goodman, M D, Williams A J, Wilson D J, Eskandari T, Vogt L M, Boland J F, Scherf U, Vockley J G. Identification of differentially expressed genes in hepatocellular carcinoma and metastatic liver tumors by oligonucleotide expression profiling. Cancer 2001; 92: 395-405.
43. Vaquerizas J M, Conde L, Yankilevich P, Cabezón A, Mínguez P, Díaz-Uriarte R, Al-Shahrour F, Herrero J, Dopazo J. GEPAS, an experiment-oriented pipeline for the analysis of microarray gene expression data. Nucleic Acids Res 2005; 33: 616-620.
44. Al-Shahrour F, Minguez P, Tárraga J, Montaner D, Alloza E, Vaquerizas J M, Conde L, Blaschke C, Vera J, Dopazo J. BABELOMICS: a systems biology perspective in the functional annotation of genome-scale experiments. Nucleic Acids Res 2007; 34: 472-476.
45. Herrero J, Valencia A, Dopazo J. A hierarchical unsupervised growing neural network for clustering gene expression patterns. Bioinformatics 2001; 17: 126-136.
46. Benjamini Y, Hochberg Y. Controlling the false discovery rate: a practical and powerful approach to multiple testing. J Royal Statist Soc B 1995; 57: 289-300.
47. Al-Shahrour F, Arbiza L, Dopazo H, Huerta J, Minguez P, Montaner D, Dopazo J. From genes to functional classes in the study of biological systems. BMC Bioinformatics 2007; 8: 114
48. Al-Shahrour F, Diaz-Uriarte R, Dopazo J. FatiGO: a web tool for finding significant associations of Gene Ontology terms with groups of genes. Bioinformatics 2004; 20: 578-580.
49. Al-Shahrour F, Minguez P, Tarraga J, Medina I, Alloza E, Montaner D, Dopazo J. FatiGO+: a functional profiling tool for genomic data. Integration of functional annotation, regulatory motifs and interaction data with microarray experiments. Nucleic Acids Res 2007; 35: 91-96.

Various modifications of the invention in addition to those shown and described herein will become apparent to those skilled in the art from the foregoing description and fall within the scope of the appended claims. The advantages and objects of the invention are not necessarily encompassed by each embodiment of the invention.

1. A method for treating preeclampsia, the method comprising:
determining whether a subject is at an increased risk of developing preeclampsia by measuring a level of annexin A2 (ANXA2) in a test sample obtained of the subject,
comparing the level of ANXA2 in the test sample to a control level of ANXA2 to determine if the subject is at an increased risk of developing preeclampsia; and
administering to the subject determined to be at an increased risk of developing preeclampsia an effective amount of a glycosaminoglycan.

2. The method of claim 1, wherein the subject has no history of preeclampsia.

3. The method of any one of claims 1-2, wherein the sample is selected from the group consisting of a sample of endometrium tissue, endometrial stromal cells and endometrial fluid.

4. The method of any one of claims 1-3, wherein the control level of ANXA2 is derived from subjects who have had a successful pregnancy and no history of preeclampsia.

5. The method of any one of claims 1-4, wherein the glycosaminoglycan is selected from the group consisting of low molecular weight heparin, heparan sulfate, chemically modified heparin or heparan sulfate, low molecular weight dermatan sulfates and mixtures thereof.

6. The method of any one of claims 1-5, wherein the level of ANXA2 is determined using an immune assay selected from the group consisting of ELISA, Western Blot, and immunohistochemical staining.

7. The method of any one of claims 1-6, wherein the subject is known to be pregnant.

8. The method of any one of claims 1-6, wherein the subject is trying to get pregnant.

9. A method for diagnosing preeclampsia or aiding in the diagnosis of preeclampsia, the method comprising:
measuring a level of annexin A2 (ANXA2) in a test sample obtained of a subject; and
comparing the level of ANXA2 in the test sample to a control level of ANXA2 to determine if the subject is at an increased risk of developing preeclampsia.

10. The method of claim 9, wherein the subject has no history of preeclampsia.

11. The method of any one of claims 9-10, wherein the sample is selected from the group consisting of a sample of endometrium tissue, endometrial stromal cells and endometrial fluid.

12. The method of any one of claims 9-11, wherein the control sample is obtained from subjects who have had a successful pregnancy and no history of preeclampsia.

13. The method of any one of claims 9-12, wherein the level of ANXA2 is determined using an immune assay selected from the group consisting of ELISA, Western Blot, and immunohistochemical staining.

14. The method of any one of claims 9-13, wherein the subject is known to be pregnant.

15. The method of any one of claims 9-13, wherein the subject is trying to get pregnant.

16. A method for treating preeclampsia, the method comprising:
obtaining an endometrial fluid sample of a subject who does not presently have preeclampsia, wherein the subject is pregnant or wherein the subject has plans to become pregnant;
performing an assay to determine level of ANXA2 in the endometrial fluid sample;
comparing the level of ANXA2 in the endometrial fluid sample to a control level of ANXA2 to determine if the subject is at an increased risk of developing preeclampsia; and
administering to the subject an effective amount of a glycosaminoglycan if the subject is determined to be at an increased risk of developing preeclampsia.

17. The method of claim 16, wherein the subject has no history of preeclampsia.

18. The method of any one of claims 16-17, wherein the control level of ANXA2 is derived from subjects who have had a successful pregnancy and no history of preeclampsia.

19. The method of any one of claims 16-18, wherein the glycosaminoglycan is selected from the group consisting of low molecular weight heparin, heparan sulfate, chemically modified heparin or heparan sulfate, low molecular weight dermatan sulfates and mixtures thereof.

20. The method of any one of claims 16-19, wherein the level of ANXA2 is determined using an immune assay selected from the group consisting of ELISA, Western Blot, and immunohistochemical staining.

21. A method for treating preeclampsia, the method comprising:
identifying a subject that has low levels of ANXA2 as compared to a control level of ANXA2, has plans to get pregnant and has no history of preeclampsia; and
administering to the subject a glycosaminoglycan in amount sufficient to raise the level of ANXA2 in the subject.

22. The method of claim 21, wherein the control level of ANXA2 is derived from subjects who have had a successful pregnancy and no history of preeclampsia.

23. The method of any one of claims 21-22, wherein the glycosaminoglycan is selected from the group consisting of low molecular weight heparin, heparan sulfate, chemically modified heparin or heparan sulfate, low molecular weight dermatan sulfates and mixtures thereof.

24. The method of any one of claims 21-23, wherein the level of ANXA2 is determined using an immune assay selected from the group consisting of ELISA, Western Blot, and immunohistochemical staining.

25. A method for assessing efficacy of glycosaminoglycan therapy for preeclampsia, the method comprising:
treating a subject who has or is at an increased risk of developing preeclampsia an effective amount of a glycosaminoglycan;
measuring levels of annexin A2 (ANXA2) in test samples obtained of the subject before and after the treatment with glycosaminoglycan, wherein an increase in the level of ANXA2 after treatment in relation to the level before treatment indicates that the glycosaminoglycan therapy is effective.

26. The method of claim 25, wherein the glycosaminoglycan is selected from the group consisting of low molecular weight heparin, heparan sulfate, chemically modified heparin or heparan sulfate, low molecular weight dermatan sulfates and mixtures thereof.

27. The method of any one of claims 25-26, wherein the level of ANXA2 is determined using an immune assay selected from the group consisting of ELISA, Western Blot, and immunohistochemical staining.
